(19) United States
(12) Patent Application Publication
(10) Pub. No.: US 2022/0047532 A1
(43) Pub. Date: Feb. 17, 2022

(54) AMPA RECEPTOR ANTAGONISTS SPECIFIC FOR CALCIUM PERMEABLE AMPA RECEPTORS AND METHODS OF TREATMENT THEREWITH

(71) Applicant: THE TRUSTEES OF THE UNIVERSITY OF PENNSYLVANIA, Philadelphia, PA (US)

(72) Inventor: Frances JENSEN, Philadelphia, PA (US)

(73) Assignee: THE TRUSTEES OF THE UNIVERSITY OF PENNSYLVANIA, Philadelphia, PA (US)

(21) Appl. No.: 17/298,599

(22) PCT Filed: Dec. 3, 2019
(86) PCT No.: PCT/US2019/064318

§ 371 Date: May 31, 2021

Related U.S. Application Data
(60) Provisional application No. 62/774,761, filed on Dec. 3, 2018.

(51) Int. Cl. A61K 31/14; A61K 31/4418; A61P 25/28; A61P 25/08

(52) U.S. Cl. CPC A61K 31/14; A61P 25/08; A61P 25/28; A61K 31/4418

(57) ABSTRACT

Antagonists that are specific for calcium permeable AMPA subtype glutamate receptors (CP-AMPARs) which lack the GluA2 subunit and methods utilizing the specific AMPA receptor antagonists to treat disorders and diseases having enhanced CP-AMPAR function or expression.

### FIELD OF THE INVENTION

The present invention provides antagonists that are specific for calcium permeable AMPA subtype glutamate receptors (CP-AMPARs) which lack the GluA2 subunit and methods utilizing the specific AMPA receptor antagonists to treat disorders and diseases having enhanced CP-AMPAR function or expression.

### BACKGROUND OF THE INVENTION

The neonatal brain exists in a heightened state of excitation, primed for activity-dependent synaptic plasticity, formation, and refinement. This hyperexcitability, although ideal for learning and memory, renders the brain vulnerable to seizures. Early-life seizures can alter development and plasticity of neuronal circuitry, which may in turn lead to cognitive impairments and autistic-like behavior. Notably, many genetic autism spectrum disorders (ASDs) exhibit early-onset seizures, indicating a convergence of cellular and molecular mechanisms in ASDs and seizure disorders. Activity-dependent synaptogenesis and downstream signaling may be an important area for therapeutic target development in treating these disorders.

Hypoxic encephalopathy, the leading clinical cause of neonatal seizures, can be refractory to conventional antiepileptic drugs and can result in later-life epilepsy, and cognitive and behavioral deficits. In our established hypoxic seizure (HS) model in P10 rats, post-seizure blockade of the AMPA receptor (AMPAR) subtype of excitatory glutamate receptors, but not NMDA receptor antagonists, prevents the long-term development of spontaneous recurrent seizures, autistic-like social behavior deficits, and synaptic plasticity deficits. In contrast to the adult, in which NMDARs primarily mediate activity-dependent dynamic Ca²⁺ signaling, the immature brain contains Ca²⁺-permeable, GluA2-lacking AMPARs, which significantly contribute to developmentally relevant intracellular signaling, such as those seen in early-life seizure models. Furthermore, L-type voltage sensitive Ca²⁺ channel (LT-VGCC) expression is also developmentally upregulated in this same period of the second postnatal week (Morton et al. Neuroscience. 2013 May 15; 238: 59-70; Morton and Valenzuela Brain Res. 2016 Feb. 15; 1633: 19-26). Given the protective effects of AMPAR blockers in our early-life in vivo seizure models, it was asked whether over-activation of CP-AMPARs and LT-VGCCs in early-life seizures might disrupt signaling pathways relevant to neurodevelopment.

The present studies examine activity-dependent phosphorylation of the transcriptional regulator methyl CpG binding protein 2 (MeCP2) as one potential pathway linking AMPARs to synaptic deficits following early-life seizures. MeCP2 plays an important role in synaptic plasticity, dendritic development, and neuronal maturation during early postnatal life, and disruptions in MeCP2 expression and function can lead to intellectual disability, autistic features, and seizure disorders, as occurs in Rett Syndrome. Neonatal seizures can also lead to autistic-like behavior, prompting the question of whether early-life seizures perturb MeCP2 function to initiate a process leading to synaptic and cognitive dysfunction.

Of the multiple phosphorylation sites that regulate MeCP2 function, S241 is primarily expressed in neuronal tissue, and regulated by activity such as Schaffer collateral stimulation. MeCP2 S421 phosphorylation is mediated by activity-dependent postsynaptic Ca²⁺ influx and CaMKII T286 phosphorylation. Prior reports implicate NMDARs and L-type voltage-gated Ca²⁺ channels (LT-VGCCs) as the primary source of Ca²⁺ mediating MeCP2 S421 phosphorylation. However, elevated expression of CP-AMPARs early in development may provide an additional route of Ca²⁺ entry into neurons. Additionally, early-life seizures potentiate Ca²⁺-dependent signaling through CP-AMPARs, activating CaMKII, PKA, PKC, and mTOR, and inducing phosphorylation of the AMPARs at GluA1 S831, GluA1 S845, and GluA2 S880. In addition, tested was whether LT-VGCC blockade would also be more effective than NMDAR blockade given their transient developmental upregulation.

As activity-dependent Ca²⁺ influx triggers MeCP2 phosphorylation, and P10 HS activate CP-AMPARs during the neonatal period, it was hypothesized that early-life seizure-induced activation of CP-AMPARs dysregulates the MeCP2 pathway, representing at least one pathway by which seizures could disrupt development of synaptic function. Presented here is evidence that in vivo neonatal seizures and in vitro neuronal depolarization induce phosphorylation of MeCP2 and its upstream activator CaMKII. Further, it is demonstrated in vivo that post-seizure blockade of AMPARs, through a novel systemically administrable CP-AMPAR inhibitor IEM-1460, prevent the dysregulation of MeCP2. Additionally, as LT-VGCCs are also transiently upregulated, it is reported that nimodipine, in vitro and in vivo can also effectively attenuate activity-dependent increases in MeCP2 S421 phosphorylation. The present results highlight potential age-specific treatment options following early-life seizures, and provide evidence for at least one pathway of overlap between early-life seizures and ASDs.

A vital need exists for treating neonates having had an early-life seizure, such as a seizure caused by hypoxic encephalopathy, wherein the neonates is a mammalian infant, including human and animal infants. There also is a critical need for treating disorders of enhanced CP-AMPAR function or expression including but not limited to epilepsy, dementia, autism, neurodevelopmental delay disorders, traumatic brain injury, and stroke. Still further a significant need remains for treating subjects having CDKL5 disorders, who suffer seizures within the first few months of life and present with developmental delay or disorders, including cognitive development.

The methods provided in the present invention administer antagonists that are specific for calcium permeable AMPA subtype glutamate receptors (CP-AMPARs) which lack the GluA2 subunit to treat a subject having an early-life seizure and disorders of enhanced CP-AMPAR function or expression, to treat disorders including but not limited to epilepsy, dementia, autism, neurodevelopmental delay disorders, traumatic brain injury, and stroke.

### SUMMARY OF THE INVENTION

In one aspect, the present invention provides an antagonist of a calcium permeable, AMPA subtype glutamate neurotransmitter receptor (CP-AMPAR), wherein CP-AMPAR lacks a GluA2 subunit.

In another aspect, the present invention provides a method for preventing or reducing the risk of developing a neurological disorder consequent to early-life seizure or hypoxic encephalopathy, the method comprising administering to a subject having had early-life seizure or hypoxic encephalopathy, an effective amount of an antagonist of CP-AMPAR, wherein CP-AMPAR lacks a GluA2 subunit.

In another aspect, the present invention provides a method for treating a subject suffering from enhanced CP-AMPAR function or expression, said method comprising administering to the subject an effective amount of an antagonist of CP-AMPAR, wherein CP-AMPAR lacks a GluA2 subunit.

In another aspect, the present invention provides a method for treating a subject suffering from a disease associated with phosphorylation of the transcriptional regulator methyl CpG binding protein 2 (MeCP2), comprising: administering an effective amount of an antagonist of a calcium permeable, AMPA subtype glutamate neurotransmitter receptor (CP-AMPAR), wherein CP-AMPAR lacks a GluA2 subunit; or an antagonist of an L-type voltage gated Ca²⁺ channels (LT-VGCC) blocker; or both.

Other features and advantages of the present invention will become apparent from the following detailed description, examples and figures. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain embodiments of the present disclosure. The AMPA receptor antagonists that are specific for calcium permeable AMPA receptors and methods of use thereof, described herein, may be better understood by reference to one or more of these drawings in combination with the detailed description of specific embodiments presented herein.

FIGS. 1A-1B show induction of phosphorylation of MeCP2 S421 and CaMKII T286 in rat cortex and increased MeCP2 S421 phosphorylation at post-seizure induction. Hypoxic seizures (HS) in P10 rats induce phosphorylation of MeCP2 S421 and CaMKII T286 in cortex. (FIG. 1A) Increased MeCP2 S421 phosphorylation at 3 hrs post-seizure induction (n=4 samples/group with 3 rat cortices pooled for one sample, p=0.003) and (FIG. 1B) increased CaMKII T286 phosphorylation at 1 hr post-seizure (n=4, p=0.009). C=normoxic controls, HS=post-seizure rats. For all figures, error bars represent standard error of mean. *p<0.05, **p<0.1, ***p<0.001, ****p<0.0001. See Suppl. Methods for HS procedure and Suppl. FIG. 1 for time course of phosphorylation relative to loading controls.

FIGS. 2A-2B demonstrate that AMPARs mediate kainic acid (KA)-induced Ca²⁺ influx in E18+10DIV cortical and hippocampal neuronal cultures. Kainic acid (KA)-induced Ca²⁺ influx in Fura-2 loaded neurons decreased following incubation with the NMDAR antagonist D-APV in C=cortical (FIG. 2A, KA+APV vs. KA, n=104 cells from 2 coverslips, p<0.0001) and (FIG. 2B) H=hippocampal neuronal cultures (KA+APV vs. KA, n=68 cells from 3 coverslips, p<0.0001). Bar graphs represent mean peak 340/380 excitation ratio, normalized to average of KA condition. NMDAR-sensitive Ca²⁺ influx was further decreased by both the AMPAR antagonist NBQX (KA+APV+NBQX, C: n=102 cells from 3 coverslips; H: n=133 cells from 5 coverslips; p<0.0001 for both) and NASPM, a blocker of Ca²⁺ permeable AMPARs (KA+APV+NASPM, C: n=133 cells from 3 coverslips; H: n=176 cells from 6 coverslips, p<0.0001 for both), normalized to average of KA+APV condition. Ratiometric scales are left of each set, scale bar=20 μm.

FIGS. 3A-3D depict induction of MeCP2 S421 phosphorylation. Kainic Acid (KA)- and KCl-induced MeCP2 S421 phosphorylation mediated by CP-AMPARs in cortical and hippocampal E18+10DIV neuronal cultures. (FIGS. 3A-3B) Immunoblots demonstrating that KA still induced MeCP2 phosphorylation in the presence of D-APV in cortex (n=4, p=0.0722) and hippocampus (n=8, p=0.8881). However, MeCP2 phosphorylation was reduced from D-APV condition by adding NBQX (C: n=4, p<0.0001; H: n=8, p<0.0001), nimodipine (C: n=4, p<0.0001, H: n=7, p<0.0001), and/or NASPM (C: n=4, p<0.0001; H: n=6, p<0.0001). Addition of nimodipine to D-APV and NASPM did not further reduce MeCP2 S421 phosphorylation in cortex (p=0.973) and hippocampus (p=0.790). (FIGS. 3C-3D) Immunoblots demonstrating that KCl-induced phosphorylation of MeCP2 S421 cannot be reversed by treatment with D-APV alone in cortex (FIG. 3C) (n=4, p=0.9637) and hippocampus (FIG. 3D) n=4, p=0.373). However, KCl-induced phosphorylation of MeCP2 S421 was reversed by NBQX alone in cortex (n=4, p=0.0069) and hippocampus (n=4, p=0.0003). MeCP2 phosphorylation was reduced from the D-APV treated condition with the addition NBQX (C: n=4, p=0.0215; H: n=4, p=0.0082), Nnmodipine (C: n=4, p=0.0005; H: n=4, p=0.0002), and NASPM (C: n=4, p=0.0241; H: n=4, p=0.0015). Addition of nimodipine to D-APV and NASPM did not significantly further reduce p-MeCP2 S421 (C: p=0.4352; H: p=0.8012).

FIGS. 4A-4B show CaMKII T286 phosphorylation is upstream of MeCP2 S421 phosphorylation and mediated by intracellular Ca²⁺ in E18+10DIV neuronal cultures. (FIGS. 4A-4B) Representative immunoblots from cortical (FIG. 4A) and hippocampal cultures (FIG. 4B) demonstrating that KA-induced phosphorylation of MeCP2 S421 is reduced by treatment with the CaMKII inhibitor KN-93 (C: n=5, p<0.0001; H: n=5, p<0.0001), but not its inactive analog KN-92 (C: n=5, p>0.9999; H: n=5, p>0.9999). MeCP2 phosphorylation is reduced by the Ca²⁺ chelator EGTA (C: n=5, p=0.0005; H: n=5, p<0.0001).

FIGS. 5A-5B demonstrate that CaMKII T286 phosphorylation is partially mediated by CP-AMPARs in E18+10DIV cell cultures. FIGS. 5A-5B, KA-induced phosphorylation of CaMKII T286 cannot be reversed by treatment with D-APV alone in cortical (FIG. 5A) (n=4, p=0.2921) and hippocampal neurons (FIG. 5B) (n=7, p=0.5213). However, CaMKII phosphorylation was reduced from the D-APV treated condition with the addition of NBQX (C: n=4, p<0.0001; H: n=7, p<0.0001), nimodipine (C: n=4 p=0.0002; H: n=6, p<0.0001), and NASPM (C: n=4, p=0.0001; H: n=6, p<0.0001). Addition of nimodipine to D-APV and NASPM did not further reduce CaMKII T286A phosphorylation in cortex (p=0.7365) or hippocampus (p=0.9290).

FIG. 6 shows AMPARs mediate Hypoxic Seizure (HS)-induced MeCP2 S421 phosphorylation in P10 rats. Increased MeCP2 S421 phosphorylation 3 hrs post-HS (HS+V: n=17 vs. C+V n=14, p=0.0003) can be attenuated by in vivo pre-treatment with the AMPAR antagonist NBQX (20 mg/kg, i.p.) (HS+NBQX: n=9, vs. HS+V, p<0.0001), the CP-AMPAR blocker IEM-1460 (20 mg/kg, i.p.) (HS+IEM-1460: n=9, vs. HS+V p=0.0099), or the LT-VGCC antagonist nimodipine (10 mg/kg, i.p.) (HS+NIM: n=9, vs. HS+V p=0.0051).

FIG. 7 shows genes that are shared between epilepsy and autism/NDD.

FIG. 8 illustrates shared signaling paths between epilepsy and autism/NDD.

FIG. 9 demonstrates that epilepsy and autism converge at the synapse.

FIG. 10 illustrates that the CDKL5 knock-in (KI) mouse has a nonsense mutation in the catalytic domain.

FIGS. 11A-11H graphically depict CDKL5 KI mice exhibit autistic-like behaviors such as deficits in social interaction, learning, and memory.

FIG. 12 graphically shows that CDKL5 KI mice display lower seizure threshold than wild type (WT) littermate controls. Mice were administered a sub-threshold dose (40 mg/kg, i.p.) of pentylenetetrazol. Mice were video-monitored for 1 hour post-injection. Seizures were scored separately by two observers using a modified Racine scale. Time to first seizure event (myoclonic jerks) was recorded.

FIG. 13 graphically illustrates maturational changes in Glutamate and GABA receptor function in the developing brain.

FIG. 14 shows that AMPAR subunit GluA2 regulates Ca²⁺ permeability and is essential for normal synaptic function. GluA2 expression low during early postnatal development and CP. Seizures decrease GluA2 expression in both immature and mature brain. GluA ser880 phosphorylation post monophasic seizure causes subunit trafficking out of membrane GluA2 KO/allelic KO mice show seizures and LTP abnormalities. Seizure induced GluA2 deficit contributes to epileptogenesis

FIGS. 15A-15C illustrate that AMPA receptor (AMPAR) subunit GluA2 is significantly decreased in membrane preparations of CDKL5 KI hippocampus. WB whole cell-prepped hippocampal tissue does not show change. Decreased GluA2:GluA1 suggests increase in GluA2-lacking, Ca²⁺ permeable AMPARs. No significant changes observed in NMDA receptor subunits in the hippocampus. No changes in AMPA or NMDA receptors were found in the cortex. No changes in AMPAR mRNA expression.

FIGS. 16A-16B show that decreases in GluA2 have been linked to hyperexcitability and altered plasticity. In WT, seizures at P10 decrease surface GluA2 (FIG. 16A) Fraction of synapses containing GluA2 puncta (% WT at m ax threshold) in CA1GluA2 and DG GluA2, respectively showing WT compared to CDKL5 KI (FIG. 16B).

FIGS. 17A-17G illustrate that CDKL5 KI mice exhibit elevated early-phase long-term potentiation (LTP) and normal long-term depression (LTD).

FIG. 18 shows that targeting Ca2+ permeable AMPARs is age and disease specific.

FIG. 19 graphically illustrates evidence for decreased GluA2 in human CDKL5.

FIG. 20 outlines the targeting of E:I imbalance for therapy.

FIG. 21A-21B show a comparison % WT mTOR, p-m TOR and p-mTOR/total in the cortex and the hippocampus of wild type (WT) and CDKL5 KI, respectively. mTOR is Mammalian Target of Rapamycin.

FIG. 22 graphically illustrates various proteins in CDKL5 disorder and in Rett syndrome (RTT) in control and disease states.

FIG. 23 ELS induce altered hippocampal CA1 AMPAR function, silent synapses, and synaptic plasticity. Hippocampal brains slices removed at 48-72 h after P10 hypoxic seizures (HS) cause (A) enhanced AMPAR-mediated sEPSCs, (B) inwardly rectifying AMPAR eEPSCs, and (C) a precocious loss of silent synapses indicated by both decreased failure rates and silent synapse fraction. These changes yield attenuated synaptic plasticity both in early-life and as adults: (D) reduced eEPSC amplitude in post-HS 48-72 hrs from whole-cell LTP pairing protocol; (E,F) decreased potentiation from extracellular LTP recordings.

FIG. 24 Post-seizure treatment with NBQX (A) attenuates the enhanced AMPAR function following ELS, (B-C) reverses premature silent synapse loss, and protects against impaired LTP at both (D) 48-72 hr post HS and (E) as P60 adults.

FIG. 25 PTZ-ELS induced thalamocortical silent synapse loss in primary auditory cortex (AI). (A) Thalamocortical slice schematic with stimulation in medial geniculate body (MGBv) and recordings in A1 L4 pyramidal neurons. (B,C) Representative eEPSCs using minimal stimulation intensity. (D,E) Lack of significant difference in eEPSC failure rates in PTZ compared to controls, summarized in (F). (G) Reduced fraction of silent synapses in PTZ group. Loss of silent synapses is associated with impaired tonotopic critical period plasticity.

FIG. 26 The CDKL5 R59X mouse model shows an overexpression of GluA2-lacking AMPAR with behavioral deficits. (A-C) Increased inwardly rectifying AMPAR eEPSCs relative to WT littermates. (D-E) Enhanced AMPAR currents are attenuated by IEM-1460 and NASPM, selective blockers of GluA2-lacking receptors. Acute in vivo treatment with IEM-1460 improves (F) spatial and temporal working memory in Y maze, indicated by % spontaneous alternating behavior and (G) autistic like impairment in social behavior.

FIG. 27 Following P10 PTZ-ELS, FosGFP mice indicate increased neuronal firing and AMPAR function selectively in GFP+ hippocampal CA1 neurons. (A) Hippocampal slices were prepared 2-3 hrs post ELS. GFP+ or GFP− pyramidal neurons were patched. (B) DIC image of acute slice. (C-F) GFP+ neurons have significantly increased amplitude & frequency in AMPAR mESPCs compared to GFP− neurons & control littermates (n=11-15). (G-I) GFP+ cells have significantly larger minimally evoked-EPSCs, and (J-L) have inwardly rectifying AMPARs eEPSCs indicating presence of GluA2-lacking AMPARs (n=9).

FIG. 28 Experimental design. (A) KA & PTZ model will be used for evaluation of hippocampus and auditory cortex, respectively. KA model: ELS will be given at P10. Mice are pre-treated with 4-OHT 1 hr prior to KA/saline injection. In Aims 1 d and 2 d, 1 hr post KA mice will receive NBQX/IEM1460/vehicle. PTZ model: the same paradigm will be followed for 3 consecutive days (P9-11) as per Sun et al, 2018. Aims 1 & 2 will evaluate the evolution of activated neurons from ELS at timepoints highlighted in red. Aim 3 will use the same ELS except a 2nd seizure (LLS) will be given at P30 or P60 and immediately euthanized 4 hrs post-seizure to utilize the transient Fos-GFP signal. (B) Schematic & chart summarizing neuronal activation. (C) Imaging of P30 KA hippocampus (scale bar: 200 μm) with subfields (100 μm).

FIG. 29 FosTRAP activation following ELS paradigms with KA & PTZ models. KA mice exhibit robust hippocampal activation with minimal cortical tdTom+; PTZ mice exhibit strong cortical activation. Little baseline activation is seen with handling (saline). Scale bar: 300 μm.

FIG. 30 FosTRAP CA1 hippocampal slice electrophysiology at P28 post-KA ELS. (A) Fluorescent tdTom overlayed on DIC image. (B) Representative traces from control no-seizure mice and tdTom+ cell from ELS FosTRAP mice. TdTom+ cells show inwardly rectifying AMPAR currents: (C) I-V plot; (D) rectification index.

FIG. 31. FosGFP+ cells activated by a single PTZ seizure have decreased CA1 silent synapses and increased AMPAR single-channel conductance compared to surround non-activated GFP− cells. (A,B) Representative traces and plots of minimally evoked-EPSCs in GFP+ and GFP− cells 2 hrs post-PTZ seizures. (CF) GFP+ cells have significantly decreased eEPSC failure rates at −60 and +40 mV, indicating a reduced fraction of silent synapses (n=6-8). (G,H) Representative AMPAR sEPSCs events and fitted curves with peaked-scaled nonstationary fluctuation analysis for GFP+ and GFP− cells. (I) GFP+ cells have significantly increased AMPAR number at a single synapse, and (J) AMPAR single-channel conductance compared to GFP− and control cells (n=6-8).

FIG. 32 PTZ seizures at P9-11 show robust tdTom+ with a stage 5 seizures. Preliminary cohort of mice (n=3/group) treated with NBQX 1 hr post PTZ stage 5 seizures show a reduction in tdTom+ cells. Scale bar: 100 μm.

FIG. 33 Altered gene expression in activated GFP+vs. non-activated GFP− neurons following PTZ seizures. (A,B) FACS used to isolated GFP+ and GFP− neurons 2 hrs post PTZ in FosGFP mice (n=4). (C-E) RT-qPCR to measure relative mRNA expression changes in cFos, GluA1 and GluA2 mRNA (n=4).

FIG. 34 LT-TISA for single cell & dendrite transcriptome isolation from fixed tissue. (A) LT-TISA probe that anneals to single-stranded RNA. Photoconvertible dideoxynucleotide Cy5 moiety at 3′ end. Upon light activation the Cy5 fluorescence is lost and the free 3′-OH formed acts as an in situ primer for cDNA synthesis. (B) Confocal images in hippocampus showing loading of LTTISA probe and tdTom expression. Top row, before UV laser activation of single tdTom+/TISA probe+ cell (arrow). Middle row, reduced fluorescent intensity of indicated cell after UV activation, quantified in (C). Bottom row, LT-TISA probes can bind RNA in dendritic processes in both tdTom+(arrow) and tdTom− cells (arrowhead). Scale bar: 20 μm.

FIG. 35 Reduced synaptic GluA2 expression in hippocampal CA1 post hypoxic ELS and increased inward rectification of AMPAR currents. (AD) Modified from Lippman-Bell et al, 206. (E) FosTRAP mouse hippocampus stained for synapsin and MAP2 and imaged in CA1 s. radiatum. Scale bar: 10 μm

FIG. 36 Effects of ELS (P10) and LLS (P30) in FosTRAP/FosGFP, euthanized 2 hrs post LLS. FosTRAP tdTom indicates cells activated by P10 seizure, and FosGFP+ cells from 2nd seizure. Scale bar: 20 μm.

### DETAILED DESCRIPTION

In the following detailed description, numerous specific details are set forth in order to provide a thorough understanding of the antagonists described herein and methods of treatment with the antagonists described herein.

In one aspect, the present invention provides an antagonist of a calcium permeable, AMPA subtype glutamate neurotransmitter receptor (CP-AMPAR), wherein said CP-AMPAR lacks a GluA2 subunit. In an embodiment, the antagonist is IEM1460. In another embodiment, the antagonist is systemically administrable.

In another aspect, the present invention provides a method for treating a subject suffering from enhanced CP-AMPAR function or expression, said method comprising administering an an effective amount an effective amount of an antagonist of a calcium permeable, AMPA subtype glutamate neurotransmitter receptor (CP-AMPAR), wherein said CP-AMPAR lacks a GluA2 subunit to the subject.

In another aspect, the present invention provides a method for treating a subject suffering from a disease associated with phosphorylation of the transcriptional regulator methyl CpG binding protein 2 (MeCP2), comprising: administering an effective amount of an antagonist of a calcium permeable, AMPA subtype glutamate neurotransmitter receptor (CP-AMPAR), wherein CP-AMPAR lacks a GluA2 subunit; or an antagonist of an L-type voltage gated Ca²⁺ channels (LT-VGCC) blocker; or both.

In another aspect, the present invention provides a method for preventing or reducing the risk of developing a neurological disorder consequent to early-life seizure or hypoxic encephalopathy, the method comprising administering to a subject having had early-life seizure or hypoxic encephalopathy, an effective amount of an antagonist of CP-AMPAR, wherein CP-AMPAR lacks a GluA2 subunit.

In an embodiment, the subject is at a developmental stage having a predominance of GluA2-lacking AMPARs. In another embodiment, the subject has an early-life seizure. In a further embodiment, the subject has hypoxic encephalopathy. In a still further embodiment, the subject has a CDKL5 disorder. In an embodiment, the subject further has one or more neurologic disorder. In various embodiments, the one or more neurologic disorder is infantile spasms, Lennox Gastaut syndrome, Rett Syndrome, West Syndrome, and autism. In another embodiment, the subject has epilepsy.

In yet another embodiment, the subject has an autism spectrum disorder. In a further embodiment, the subject has dementia. In a still further embodiment, the subject has a neurodevelopmental delay disorder. In a separate or related embodiment, the subject has a traumatic brain injury. In another embodiment, the subject has a stroke. In a further embodiment, the seizure is post-natal. In a still further embodiment, the seizure is from 3 to 6 months after birth. In an embodiment, the antagonist is administered from between immediately post-seizure to 6 months post-seizure. In another embodiment, the antagonist is administered immediately post-seizure.

In a further embodiment, the method further comprising administering an L-type voltage gated Ca²⁺ channels (LT-VGCC) blocker. In an embodiment, the LT-VGCC blocker is nimodipine.

In another embodiment, the administration of the antagonist either delays later-life epilepsy. In yet another embodiment, the administration of the antagonist further either delays or reduces incidence of later-life epilepsy. In a further embodiment, the administration of the antagonist further delays or reduces incidence of autism spectrum disorders.

Any patent, patent application publication, or scientific publication, cited herein, is incorporated by reference herein in its entirety.

The following examples are presented in order to more fully illustrate the cell sheets, methods of making the sheets, and uses described herein.

### EXAMPLES

Materials and Methods

In Vivo and In Vitro Immunoblotting

For in vivo studies, male Long-Evans rats were sacrificed at 0.5, 1, 3, 6, and 24 hr following hypoxia-induced seizures (along with normoxic littermate controls) at P10 (see Supp. Methods), and given an intraperitoneal injection of either 20 mg/kg NBQX (Sigma, saline), 20 mg/kg IEM-1460 (Tocris, saline), 5 or 10 mg/kg nimodipine (Sigma, 10% DMSO/50% polyethylene glycol/40% dH₂O mixture), or vehicle within 30 min after hypoxic seizures. Nuclei were isolated as previously described (81). For in vitro studies, 10DIV cells were prepared from Long Evans E17/18 rats (plated at 1-1.5×10⁶ cells/well in 6 well plates) and treated for 2 hr with 1.1M tetrodotoxin (TTX, Tocris), 100 μM D-APV (Tocris), 5004 NBQX (Tocris), 15004 NASPM (Sigma), 5 μM nimodipine (Tocris), 5 μM KN-92 (Calbiochem), 5 μM KN-93 (Calbiochem), and/or 1 mM EGTA (Sigma). Neurons were then stimulated for 1 hr with 100 μM KA (Tocris) or 55.04 KCl (Sigma). Blots were incubated with the following primary antibodies: MeCP2 (S421) (1:1000) and total-MeCP2 (1:1000, kind gifts from Dr. Michael Greenberg, Harvard Medical School), phospho-CaMKII (T286) (1:250, Cell Signaling), pan-CaMKII (1:250, Cell Signaling), (3-actin (1:5000, Sigma), and lamin A/C (1:1000, Cell Signaling).

Calcium Imaging

Ratiometric Ca²⁺ imaging was performed on E17/18+10 DIV hippocampal and cortical cultures, plated at 1×10⁵ cells/well in 24 well plates. Cultures were pretreated for 2 hr with 1 μM tetrodotoxin (TTX, Tocris), loaded with 15-20 μM Fura-2 AM (Invitrogen) and 0.1% pluronic F-127 (Invitrogen) for 30 min, then washed for 30 min (all solutions hereafter were made in warmed, oxygenated ACSF containing 1 μM TTX). After 10 min bathed in 100 μM D-APV (Tocris), cells were stimulated using fast (<2 min) bath application of 30 μM kainate (KA). After washout, cells were treated for 10 min with D-APV plus 50 μM NBQX or 150 μM 1-Napthylacetyl Spermine (NASPM, Sigma), then stimulated with KA again. Changes in Ca²⁺ influx were assessed by change in 340/380 nm excitation ratio from baseline in individual somas (using regions of interests) in NisElements software (see Supp. Methods).

Statistical Analysis

Group data were expressed as mean±SEM, with n representing the number of rats for a given data point (in vivo) or coverslips (in vitro), unless stated otherwise. Ca²⁺ imaging experiments were analyzed via paired 2-way student's t-test or 1-way ANOVA. For multiple comparisons across >2 conditions, one-way ANOVA followed by post hoc Tukey's or Bonferroni multiple comparison tests was used. For the in vivo post-seizure time-course experiments, two tailed t-tests corrected for multiple comparisons with the Holm-Sidak method were used. Statistical significance was defined as p<0.05.

The present disclosure is not limited to the drawings or to the corresponding descriptions. Meanings of technical and scientific terms used herein are to be commonly understood as by one of ordinary skill in the art to which the disclosure belongs, unless otherwise defined. While the certain features have been described with respect to a limited number of embodiments, these should not be construed as limitations on the scope of the disclosure, but rather as exemplifications of some of the embodiments. Other possible variations, modifications, substitutions, changes, and equivalents are also within the scope of the disclosure. Accordingly, the scope of the disclosure should not be limited by what has thus far been described, but by the appended claims and their legal equivalents.

### Example 1

### In Vivo Hypoxic Seizures (HS) in P10 Rats Induce Phosphorylation of MeCP2 S421 and CaMKII T286

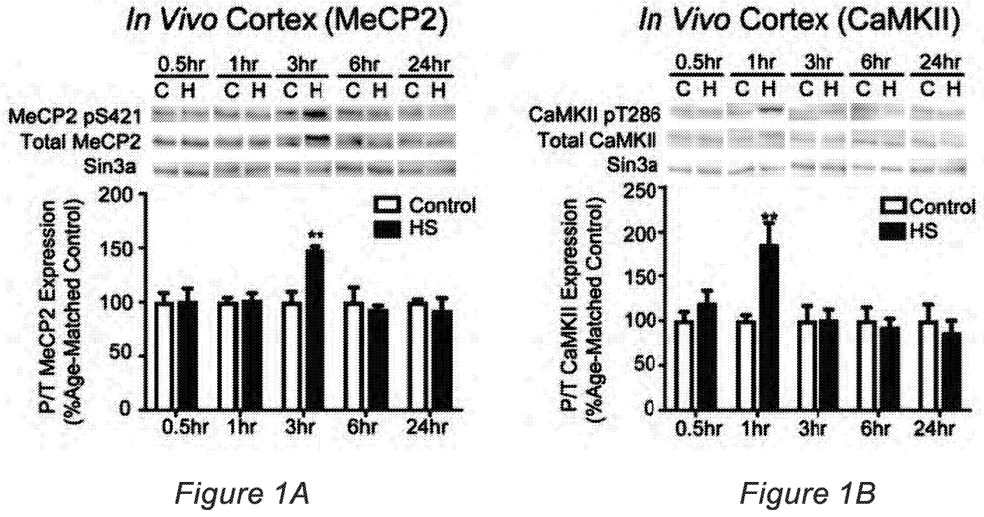

Hypoxic seizures (HS) in neonatal rats (P10) induce rapid post-translational modifications and synaptic accumulation of AMPARs (41, 44) and dysregulate several intracellular signaling cascades (21). It was hypothesized that HS at P10, an age with high levels of CP-AMPARs (22), would increase phosphorylation of MeCP2 and its upstream activator CaMKII. Indeed, cortical tissue removed post-HS showed a significant increase in MeCP2 S421 phosphorylation at 3 hrs post-HS (Suppl. FIG. 1, FIG. 1A: 148±11% vs. normoxic controls, p=0.003), and elevated CaMKII T286 phosphorylation at 1 hr post-HS (FIG. 1B: 184±20% p=0.009). These findings indicate that in vivo early-life seizures induce transient activity-dependent regulation of CaMKII and MeCP2.

### Example 2

### Identification of Functional CP-AMPARs at E18+10DIV

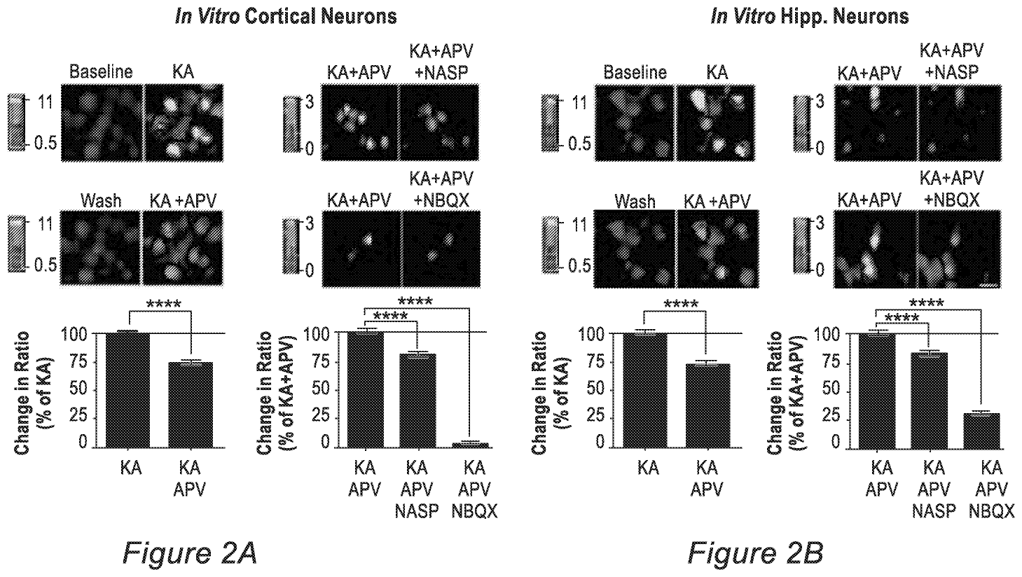

To determine whether CP-AMPARs facilitate HS-mediated MeCP2 and CaMKII phosphorylation in early life, their role in activity-dependent signaling in cortical and hippocampal primary neurons in vitro was examined. It was hypothesized that GluA2-lacking CP-AMPARs in young (E18+10DIV) neuronal cultures (45) and early postnatal rodent and human brains in vivo (22, 23) would provide an added source of Ca²⁺ to supplement signaling through NMDARs and VGCCs reported previously in more mature neurons (29). To confirm the presence of CP-AMPARs, neuronal Ca²⁺ influx via Fura-2 Ca²⁺ imaging in E18+10DIV cultured cortical and hippocampal neurons stimulated by kainic acid (KA, 30 μM) was measured. KA-induced Ca²⁺ influx was reduced by the NMDAR antagonist D-APV (100 μM), suggesting that NMDA receptors contribute to about 25% of Ca²⁺ influx (FIG. 2A-B, normalized mean peak ratio in cortex (C): 100±2% KA only vs. 74±3% KA+APV, p<0.0001; and hippocampus (H): 100±3% KA only vs. 73±3% KA+APV, p<0.0001). The remaining NMDAR-insensitive Ca²⁺ influx (FIG. 2A,B, KA+APV C: 100±3%, H: 100±3%) was further decreased by both the AMPAR antagonist NBQX (50 μM) (C: 3±2%, H: 31±2%, vs. KA+APV condition, p<0.0001) and 150 μM NASPM, a specific blocker of Ca²⁺ permeable, GluA2-deficient AMPARs (C: 79±3%, H: 84±3%, p<0.0001). Control neurons responded equally to dual KA stimulation (Suppl. FIG. 2A), excluding the possibility that reduced Ca²⁺ influx was due to repeated KA stimulation. Together, these findings confirm that CP-AMPARs facilitate part of the non-NMDAR-mediated Ca²⁺ influx at E18+10DIV.

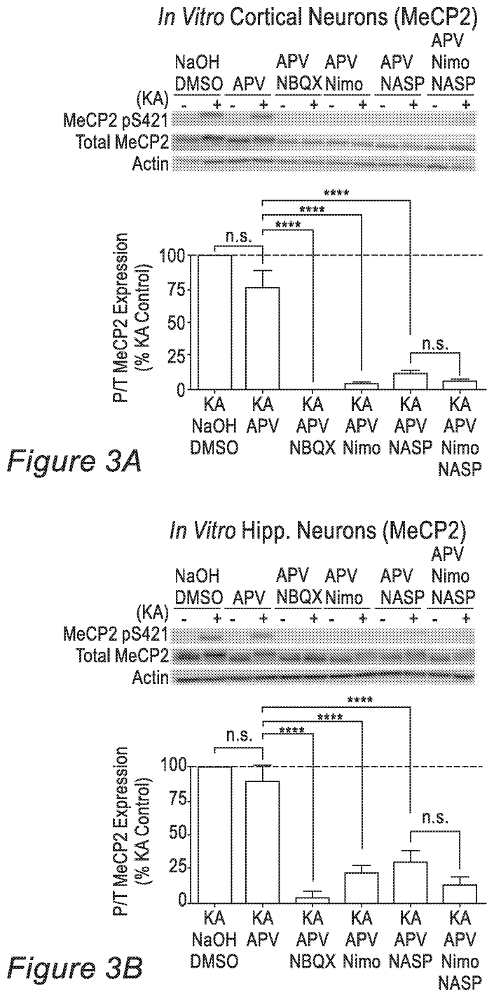

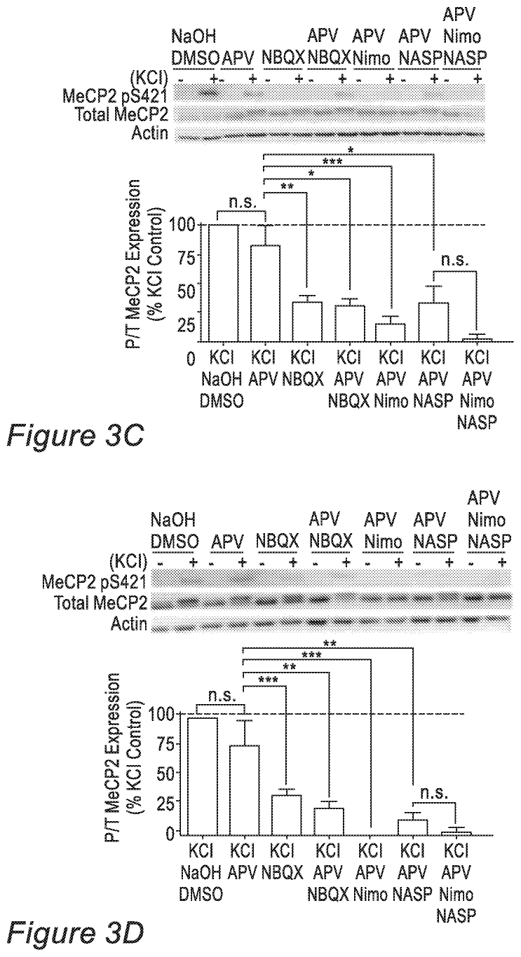

Given the critical role of the GluA2 subunit in regulating Ca²⁺ permeability, immunocytochemistry was used to measure the percentage of GluA1 subunits colocalized with GluA2. Consistent with prior reports (45-47), only about 31±4% of GluA1-containing AMPARs were colocalized with GluA2 at the earlier E17/18+10DIV time point vs. 61±2% by 13DIV (Suppl. FIG. 3, p<0.0006), suggesting a predominance of GluA2-lacking AMPARs at this developmental stage, supported by in vivo observations in intact developing brain (24). Combined with Ca²⁺ imaging, this suggests that GluA2-lacking CP-AMPARs in early life may mediate activity-dependent Ca²⁺ signaling.

### Example 3

### CP-AMPARs Mediate MeCP2 Phosphorylation in E18+10DIV Cell Cultures

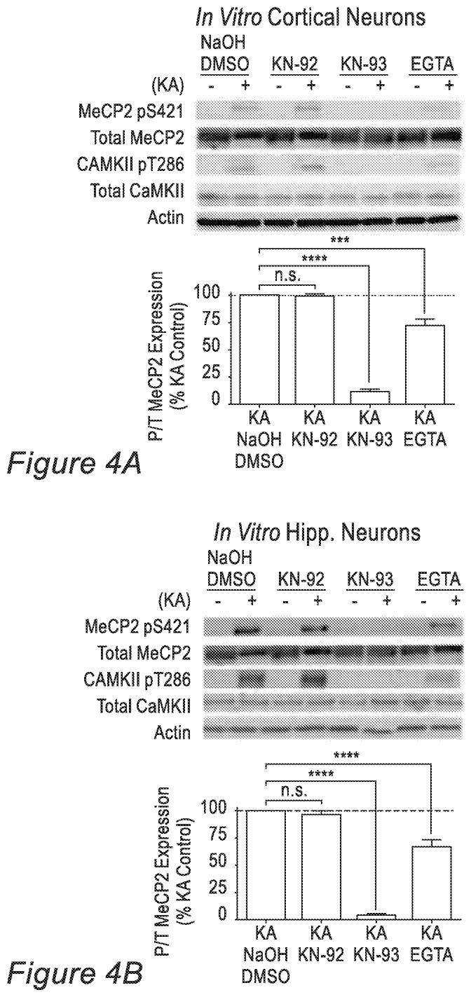

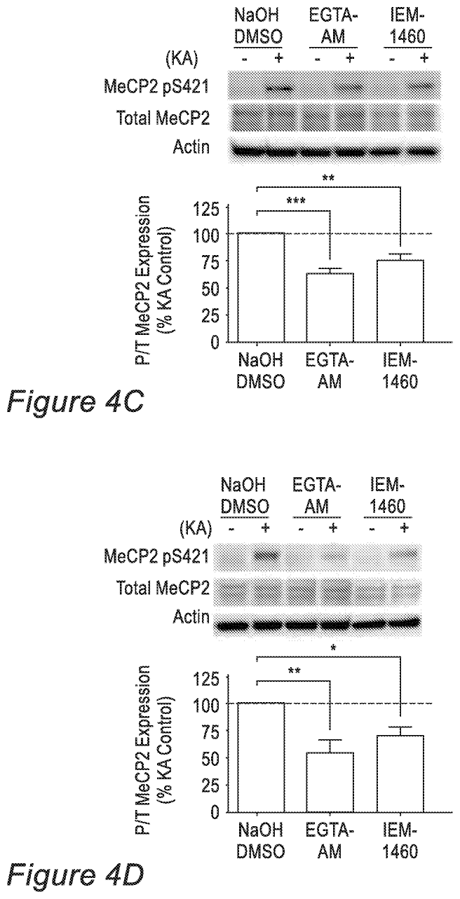

The role of CP-AMPARs in activity-dependent MeCP2 phosphorylation in vitro. was investigated. It was predicted that elevated AMPAR-mediated Ca²⁺ influx at E18+10DIV, an age analagous to P10 in rodents or term to infancy in humans (1), may provide age-specific seizure-induced hyperphosphorylation of MeCP2. First kainic acid (KA, 100 μM) stimulation was used to target ionotropic glutamate receptors and isolate the effects of upstream mediators on MeCP2 phosphorylation in E18+10DIV cultures (Suppl FIG. 4). As in older (12DIV) cultures (29), synaptic stimulation induced MeCP2 S421 phosphorylation in both cortical (FIG. 3A) and hippocampal neurons (FIG. 3B). However, unlike prior studies, 100 μM D-APV was not sufficient to prevent MeCP2 phosphorylation in DIV10 cortical neurons (Ctx: FIG. 3A: 76±13% vs. KA, NaOH+ DMSO treated controls, p=0.072) or hippocampal neurons (H: FIG. 3B: 89±13%, p=0.888), suggesting that MeCP2 phosphorylation requires additional Ca²⁺ influx from alternate, non-NMDAR sources. In the presence of APV, MeCP2 phosphorylation was reduced by administration of either 5004 NBQX (Ctx: FIG. 3A: 0±0.3%, p<0.0001; H: FIG. 3B: 3±5%, p<0.0001), or the CP-AMPAR antagonist NASPM (150 μM) (Ctx: 12±2%, p<0.0001; H: 30±9%, p<0.0001).

With respect to the relative efficacy of LT-VGCCs in MeCP S421 phosphorylation, it was found that nimodipine (5 μM, with D-APV) also significantly decreased MeCP2 phosphorylation following KA-induced depolarization (Ctx: FIG. 3A: 4±1%, p<0.0001; H: FIG. 3B: 21±6%, p<0.0001). However, addition of nimodipine to D-APV and NASPM did not add any additional reduction of MeCP2 S421 phosphorylation to D-APV and NASPM alone in cortex (p=0.973) and hippocampus (p=0.790). Together, these findings suggest that both CP-AMPARs and LT-VGCCs each mediate essential Ca²⁺ influx to mediate MeCP2 S421 phosphorylation in early life.

To examine the upstream mediators of Ca²⁺ influx in generalized neuronal depolarization, high stimulation using KCl was utilized and similar pharmacologic blockades as above were performed. D-APV alone did not reverse KCl-mediated MeCP2 phosphorylation in cortical (FIG. 3C: 87±21%, p=0.9637) or hippocampal neurons (FIG. 3D: 73±21%, p=0.373) at 10 DIV. In contrast, phosphorylation was reduced (in the presence of D-APV) by NBQX (Ctx: FIG. 3C: 35±5%, p=0.0215; H: FIG. 3D: 21±6%, p=0.0082), NASPM (Ctx: FIG. 3C: 32±13%, p=0.0241; H: FIG. 3D: 11±6%, p=0.0015), and nimodipine (Ctx: FIG. 3C: 10±6%, p=0.0005; H: FIG. 3D: 0±1%, p=0.0002). Nipodipine in the presence of NASPM and D-APV did not reveal and further reduction in MeCP2 phosphorylation over CP-AMPARs+NMDARs alone (Ctx: p=0.4352; H: p=0.8012); no change was found. Overall, these results indicate that both CP-AMPARs and LT-VGCCs collectively contribute to MeCP2 S421 phosphorylation via two parallel pathways leading to depolarization-induced Ca²⁺ influx.

### Example 4

### CaMKII T286 Phosphorylation in E18+10DIV Cultures is Upstream of MeCP2 Phosphorylation and Mediated by CP-AMPARs

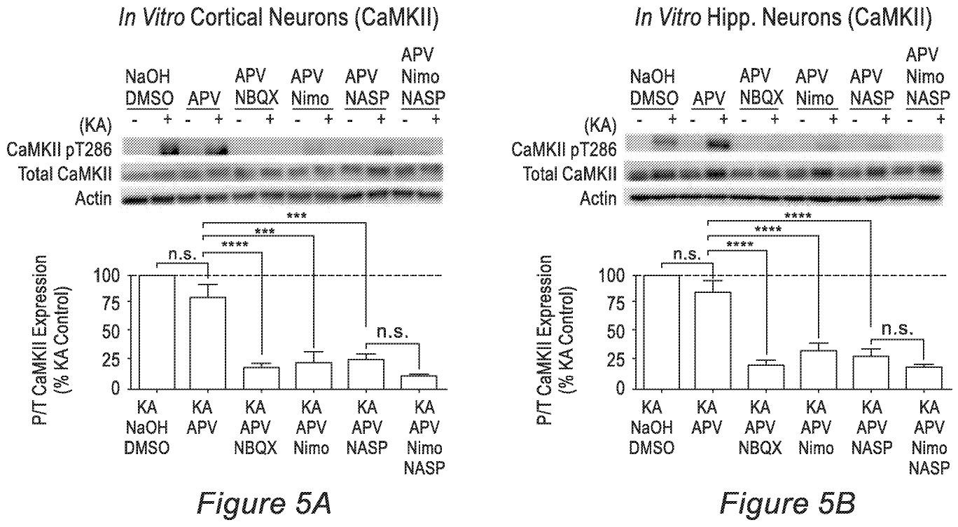

Prior studies indicate that phosphorylation of MeCP2 S421 requires both CaMKII T286 phosphorylation and intracellular Ca²⁺ influx (29), supported by the in vivo findings herein of sequential, seizure-induced activation of CaMKII and MeCP2 (FIG. 1). Consistent with these results, in both cortical (FIG. 4A) and hippocampal E18+10DIV cultures (FIG. 4B), phosphorylation of MeCP2 S421 was blocked by 5 μM KN93, a potent inhibitor of CaMKII (Ctx: 10±3%, p<0.0001; H: 3±1%, p<0.0001), but not its inactive form, KN92 (5 μM) (Ctx: 99±3%, p>0.9999; H: 97±3%, p>0.9999). Additionally, the Ca²⁺ chelator EGTA (1 mM) reduced phosphorylation of MeCP2 S421 (Ctx: 72±7%, p=0.0005; H: 66±7%, p<0.0001), as did the membrane-permeable form, 100 μM EGTA-AM (Suppl. FIG. 5, Ctx: p=0.0001, H: p=0.0057). Thus, CaMKII and intracellular Ca²⁺ regulate MeCP2 S421 phosphorylation at E18+10DIV.

As CaMKII is upstream of MeCP2 phosphorylation, we hypothesized that CaMKII pT286 required Ca²⁺ influx through the same channels as MeCP2 S421 phosphorylation. As with MeCP2, pre-treatment with D-APV did not fully reverse KA-induced CaMKII phosphorylation (Ctx: FIG. 5A: 81±11%, p=0.2921; H: FIG. 5B: 86±9%, p=0.5213). However, CaMKII phosphorylation was significantly reduced (in the presence of D-APV) with the addition of NBQX (Ctx: FIG. 5A: 18±4%, p<0.0001; H: FIG. 5B: 21±3%, p<0.0001), NASPM, (Ctx: FIG. 5A: 25±5%, p=0.0001; H: FIG. 5B: 28±7%, p<0.0001), and nimodipine (Ctx: FIG. 5A: 23±9%, p=0.0002; H: FIG. 5B: 33±6%, p<0.0001). However, LT-VGCC blockade did not reduce CaMKII T286 phosphorylation more than NASPM+D-APV alone (Ctx: p=0.7365; H: p=0.929). These results demonstrate that Ca²⁺ entry through both CP-AMPARs and L-Type VGCCs contribute to activity-dependent CaMKII T286 phosphorylation in immature cortical and hippocampal neurons, consistent with the effects on MeCP2 S421 phosphorylation.

### Example 5

In Vivo HS-Induced Phosphorylation of MeCP2 S421 is Prevented by Treatment with AMPAR Antagonists and LT-VGCC Blockade

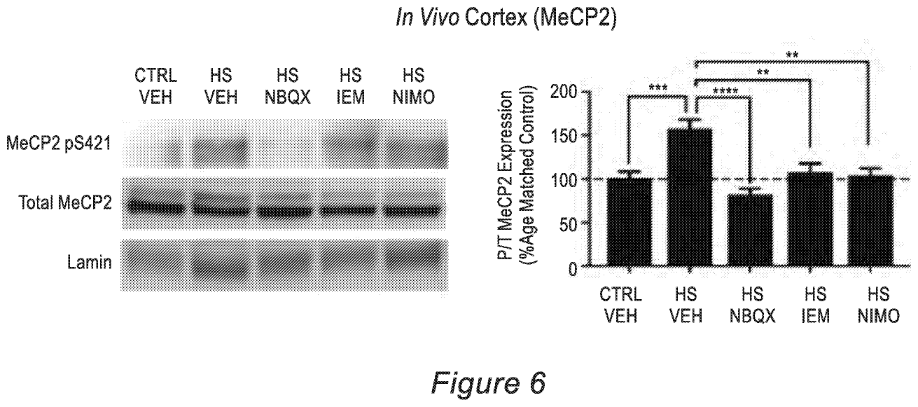

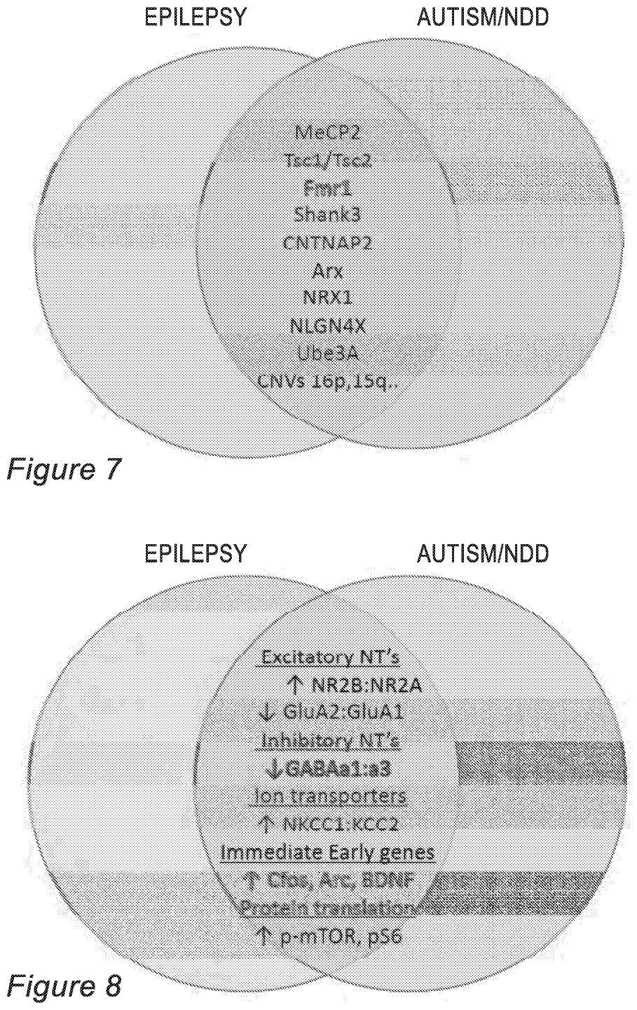

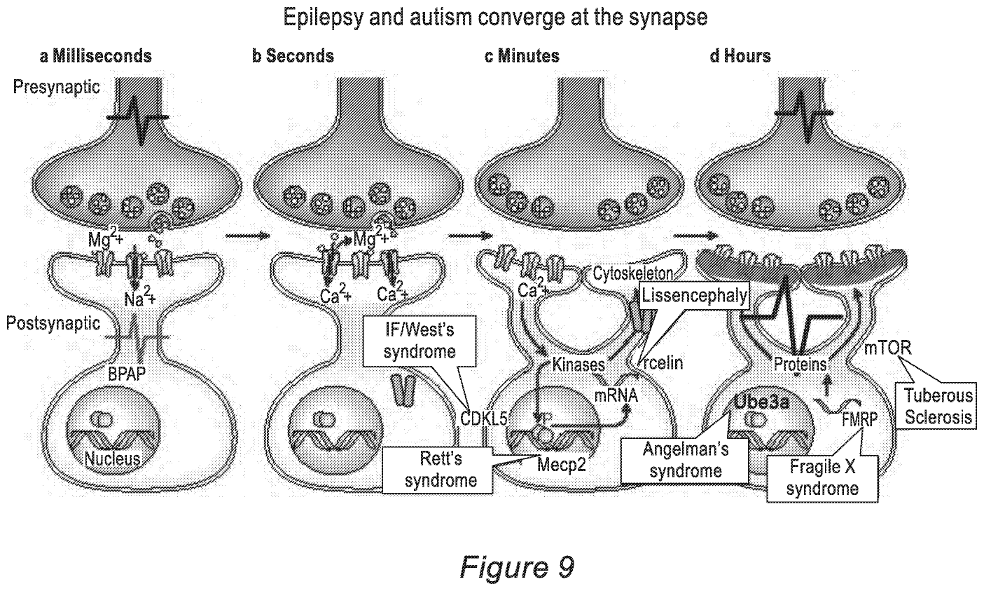

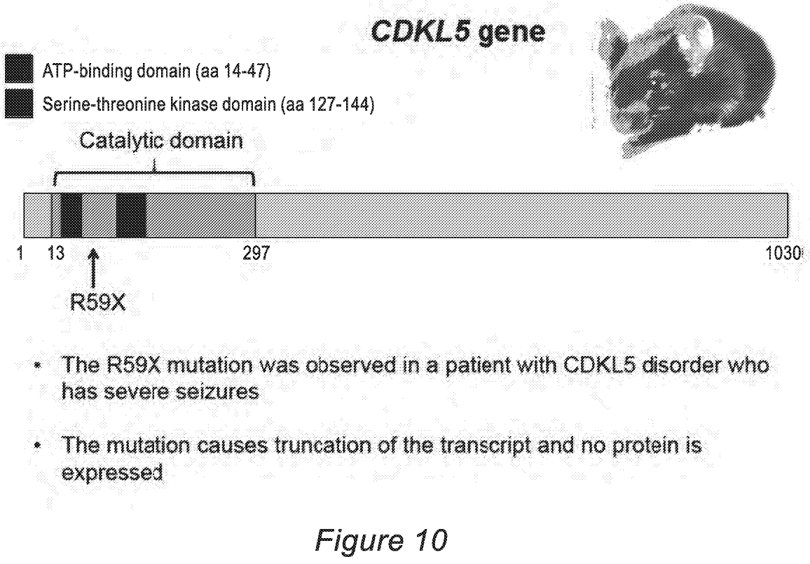

It was previously shown that post-seizure in vivo treatment with NBQX prevents later-life seizures, altered synaptic plasticity, and autistic-like social deficits (6, 12), whereas NMDAR antagonists do not prevent consequences of HS at this developmental time point (48, 49). Given the in vitro evidence for CP-AMPARs in dysregulating MeCP2, examined were the effects of NBQX and the systemically administrable CP-AMPAR-specific inhibitor IEM-1460 (43) on MeCP2 S421 phosphorylation in vivo, immediately post-HS. Consistent with the prior in vivo post-HS outcomes (12, 41, 42), administration of both 20 mg/kg NBQX and 20 mg/kg IEM-1460 (i.p.) (43, 50) significantly attenuated the increased MeCP2 S421 phosphorylation 3 hrs post-HS (FIG. 6: Control (C)+Vehicle (V) 100±8%, HS+V 157±11% normalized to mean C+V control, HS+NBQX 81±8%, HS+IEM-1460 106±11%; ANOVA p=0.002, with post-hoc Tukey's comparisons: C+V vs. HS+V p=0.0003, HS+V vs. HS+NBQX p<0.0001, HS+V vs. HS+IEM-1460 p=0.0099). It additionally was confirmed that IEM-1460 reduced activity-dependent MeCP2 phosphorylation in cultured neurons in vitro (Suppl. FIG. 5, Ctx: p<0.0049, H: p<0.0361). Paralleling the in vitro results, in vivo treatment with nimodipine reduced post-HS MeCP2 phosphorylation at 10 mg·kg, i.p. (FIG. 6: HS+NIM 103±9%, HS+V vs. HS+NIM p=0.0051), but not at a previously reported anti-convulsive dose of 5 mg/kg i.p. (Suppl. FIG. 6B) (51-53). In vivo NBQX, IEM-1460, and NIM administration did not affect baseline MeCP2 phosphorylation in control rats (Suppl. FIG. 6A). Collectively, these in vivo findings support a critical role for CP-AMPARs and LT-VGCCs in mediating seizure-induced MeCP2 phosphorylation during early development.

### Discussion

In early life, both seizures and autism share a developmental dysregulation of synaptogenesis and destabilized synaptic function, representing a potential overlap of underlying cellular mechanism. Early-life seizures contribute to later-life epilepsy and associated autistic-like behavioral deficits, and epilepsy and autism often co-occur in neurodevelopmental disorders such as Rett Syndrome. These findings suggest that seizures may dysregulate development in part through key activity-dependent neurodevelopmental signaling molecules implicated in ASDs such as MeCP2. As neuronal activity modulates MeCP2 function in part via phosphorylation of its S421 site, and neonatal seizures disrupt AMPAR and CP-AMPAR signaling, the primary aim of this study was to determine if early-life seizures could perturb MeCP2 in part through activation of Ca²⁺ via CP-AMPARs. A novel finding in this study is that neonatal seizures alter MeCP2 activity in vivo in a model previously shown to cause development of spontaneous recurrent seizures, impaired LTP, and autistic-like behavior. In addition, unlike mature neurons neurons, NMDARs do not fully block S421 phosphorylation, and both Ca²⁺-permeable AMPARs and L-type VGCCs mediate a NMDAR-insensitive component of activity-dependent MeCP2 phosphorylation in younger animals.

At P10, the rat brain is in a period of heightened synaptic development (1). One feature of this developmental stage is heightened expression of CP-AMPARs compared to adults, likely contributing to the synaptic plasticity that allows for synaptic refinement. The elevated CP-AMPAR expression may provoke the progression of epileptogenesis at this stage, as the AMPAR antagonist NBQX, but not NMDAR antagonists, prevents increased seizure susceptibility following HS at P10. To examine the mechanisms underlying activity-dependent activation of CP-AMPARs in early life, we used E18+10DIV cultured neurons, which express a high level of CP-AMPARs and MeCP2. Unlike previous studies suggesting a dominance of NMDAR-mediated MeCP2 phosphorylation in older (E18+12DIV) neuronal cultures (29), we found that two days earlier, at E18+10DIV, CP-AMPARs contribute to Ca²⁺ influx for activity-dependent MeCP2 phosphorylation. Interestingly, during this same time period, LT-VGCC expression is also developmentally upregulated. The results herein suggest that that CP-AMPARs, as well as LT-VGCCs, provide an additional critical route for activity-induced Ca²⁺ influx beyond that of the NMDARs alone in the mature brain, (perhaps due to enhanced spatiotemporal resolution for precisely timed CaMKII activation, nuclear translocation, and MeCP2 phosphorylation).

Comparing the in vivo with the in vitro xtuies here, it is important to note that our in vitro findings obtained using bath application of KA or KCl may not demonstrate the same activation and time course as synaptically released glutamate in the HS model. Supporting the in vitro studies, both the AMPAR antagonist NBQX and the novel CP-AMPAR open-channel blocker IEM-1460 prevented seizure-induced MeCP2 and CaMKII phosphorylation in vivo. Increased MeCP2 phosphorylation at 3 hrs post-HS is consistent with prior reports of CaMKII phosphorylation at 1 hr post-HS at P10 and within 1 hr post-bicuculline-mediated action potential-bursting. The in vivo efficacy of AMPAR antagonists are also reinforced by anti-seizure effects of IEM-1460 in pentylenetetrazol (PTZ)-induced seizures.

Additionally, in vivo blockade of LT-VGCCs via nimodipine at higher (10 mg/kg), but not lower (5 mg/kg) doses reduced HS-induced MeCP2 phosphorylation. Our in vitro findings suggest that Ca²⁺ influx from VGCCs and CP-AMPARs both individually contribute to MeCP2 phosphorylation, and both pathways likely converge on a common endpoint. Notably, LT-VGCCs are developmentally upregulated during early postnatal development (REFS), and have been implicated in promoting both epileptogenesis and ASD-like behavior. Ca²⁺ influx-promoting mutations in LT-VGCCs cause Timothy Syndrome, a rare genetic form of ASD. In addition to describing a new developmentally-regulated target for MeCP2 activation, CP-AMPARs, we also suggest that LT-VGCCs provide an alternative to MeCP2 phosphorylation above that of NMDARs alone.

MeCP2 likely plays a pivotal role in regulating activity-dependent gene transcription important for neural function, maturation of spine density, neuronal connectivity, dendritic arborization, behavior, and multiple forms of synaptic plasticity, including synaptic scaling and Hebbian plasticity. Mouse models of Rett Syndrome with decreased or blocked expression of MeCP2 show impaired LTP in area CA1 of the hippocampus, changes in glutamate receptor expression including decreased NR2A, behavioral deficits, and increased seizure susceptibility. All of these changes also occur in the HS neonatal seizure model, which we now show alters MeCP2 S421 phosphorylation. The phenotypic and molecular overlap between HS and MeCP2 KO mice raises the important possibility that seizures themselves impair synaptic function in part by dysregulating physiologic MeCP2 function in the immature brain. We predict that altered MeCP2 function could in turn lead to the development of autistic or cognitive behavioral deficits, similar to those seen in Rett Syndrome, presenting an area of future study. As prior reports suggest that MeCP2 serves as a transcriptional repressor, activator, or global chromatin regulator, seizure-induced changes in MeCP2 phosphorylation may have far reaching, complex effects on downstream immediate early gene and neurotrophin expression.

The present study provides the first evidence of AMPARs as a key upstream mediator of MeCP2 phosphorylation, complementing prior evidence of AMPARs as a key downstream component of MeCP2-mediated signaling. Indeed, GluA1 and GluA2 subunit trafficking is impaired both in LTP in MeCP2 KO mice, and homeostatic synaptic scaling with MeCP2 knockdown. Furthermore, AMPAR blockade prevents the post-seizure synaptic and behavioral changes reported in the HS neonatal seizure model, which are similar to the changes seen with MeCP2 KO mice. Taken together, these data suggest that AMPARs play a key role in MeCP2-regulated signaling and may thus provide therapeutic value. This idea is strongly supported by the results of the current study, in which a novel, age-specific role for CP-AMPARs in regulating MeCP2 was demonstrated, observed with both NBQX and specific blockers of CP-AMPARs, NASPM and IEM-1460. Importantly LT-VGCCs are also developmentally overexpressed and also control the NMDA-insensitive component of S421 phosphorylation reported here. The AMPAR-MeCP2 pathway, along with that of the LT-VGCCs, may provide a mechanism for post-HS long-term behavioral changes, presenting an exciting area of further research relating to the interaction between epilepsy and autism in early postnatal brain development.

### Example 6

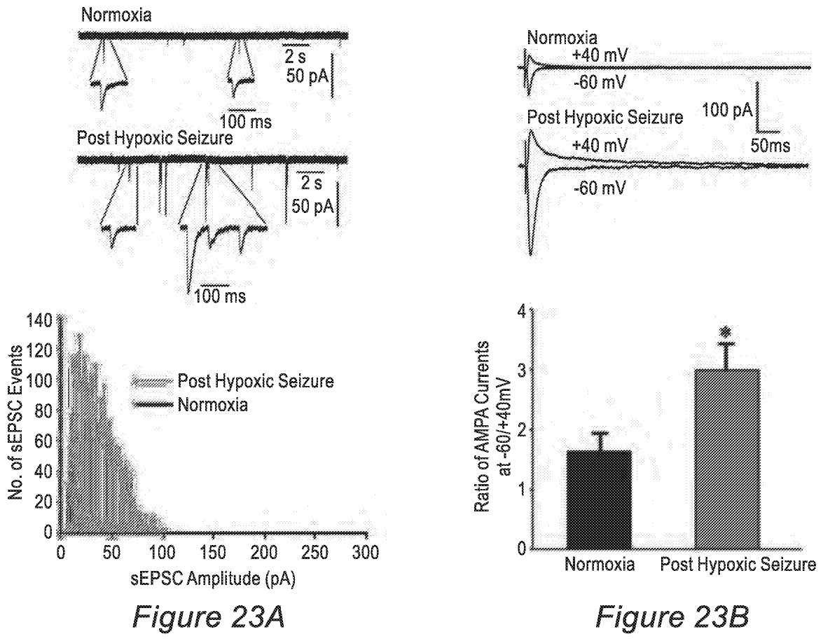

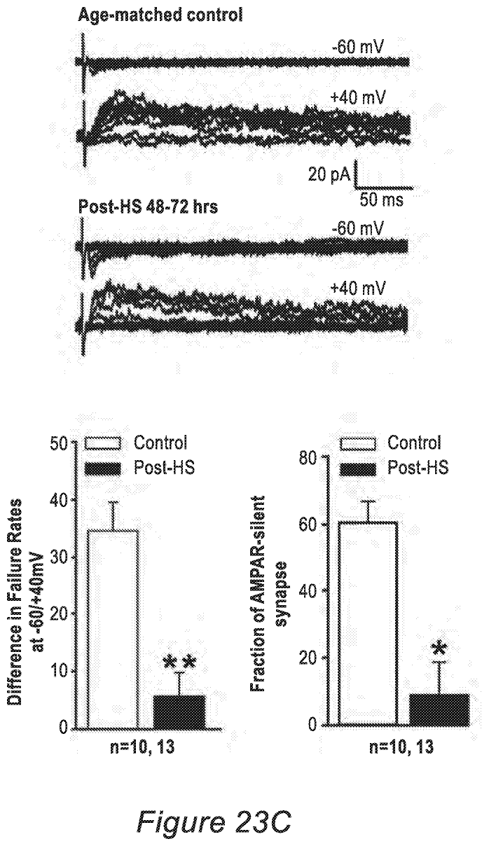

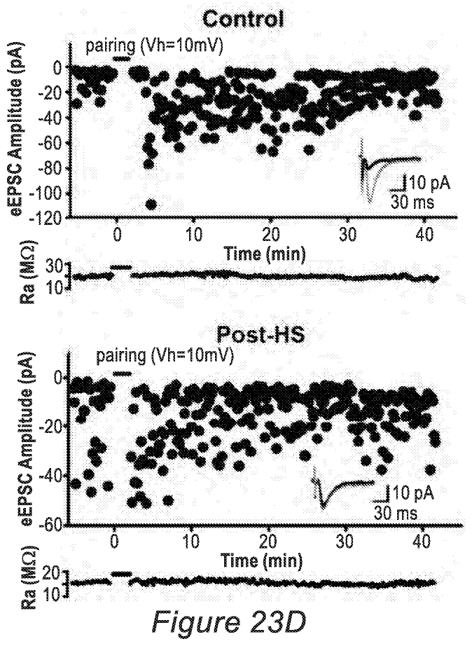

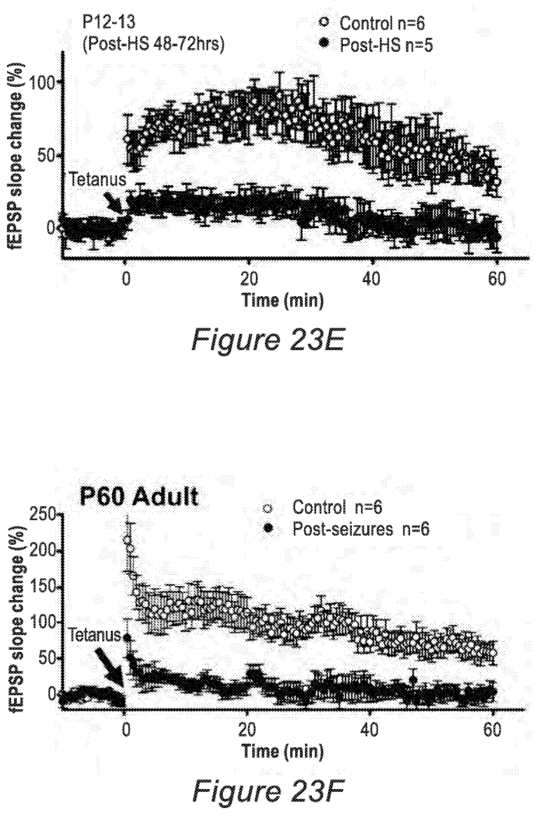

Background: Clinically, early life seizures have been associated with later intellectual disability as well as autism, and later life epilepsy. To date, there are still no specific treatments aimed at the cognitive comorbidities of early life seizures and epilepsy. Indeed, a bidirectional relationship between epilepsy and autism has been suggested, given that up to 40% of children with autism and intellectual disability also suffer from epilepsy, and approximately 35% of children with infantile spasms develop long-term intellectual disabilities including autism.86, 97 While there are multiple autism-linked genes that associate highly with epilepsy, these two disorders may also be co-acquired as a result of early life brain injury and seizures. Emerging experimental data suggest that seizures cause long lasting changes in the excitatory: inhibitory (E:I) balance, and result in cognitive and behavioral deficits, including those in social behavior with autistic-like patterns. The immature brain is relatively hyperexcitable compared to the adult due to an E:I imbalance in neuronal circuits, thought to be necessary for enhanced synaptic plasticity and learning, which characterizes the “critical period” of development. Clinical and animal model data show an exaggerated or prolonged E:I imbalance in autismin patterns that are similar to those seen in early life epilepsy, suggesting an intersection point for autism and epilepsy. Using rodent models, we and others have shown that ELS can be associated with impaired critical period synaptic plasticity, autistic-like behavioral deficits, and spontaneous seizures later in life. These models can help us understand how seizure activity and hyperexcitable networks dysregulate synaptic plasticity and necessary to develop new therapeutic strategies in this clinical space where no current cure exists. The role of AMPA-subtype glutamate receptors (AMPARs) in early life and their dysregulation by ELS. The normal critical period E:I imbalance is in part due to the maturational overexpression of both AMPA and NMDA subtypes of excitatory glutamate receptors. In early life, experience-dependent synaptic plasticity requires calcium (Ca²⁺)-activated signaling pathways inducing transcription, translation and/or post-translational changes. NMDA receptors (NMDARs) are permeable to Ca²⁺ throughout life.95 In contrast, heteromeric AMPARs are predominantly Ca2+-permeable in the immature brain, due to a developmental lack of the GluA2 subunit relative to the adult.77 GluA2-lacking receptors have higher conductance compared to GluA2-containing, and may contribute to the normal E:I imbalance of the immature brain. Despite their likely role in developmental plasticity, the reappearance of GluA2-lacking receptors in the mature brain has been linked to pathologic states such as epilepsy, autism, ischemia and addiction. The effects of ELS have been studied using a variety of seizure stimuli in rodents, including hypoxia and the chemoconvulsants pentylenetetrazole (PTZ) and kainate (KA). ELS can result in impaired later life learning and/or autistic-like social deficits and increased seizure susceptibility. We demonstrated preferential efficacy of AMPAR antagonists in suppressing ELS compared to NMDAR antagonists or inhibitory GABA receptor agonists. ELS itself alters AMPARs by inducing early activity-dependent post-translational trafficking of the AMPAR GluA1 subunit to the synapse resulting in increased spontaneous and miniature EPSC amplitude (FIG. 23A), and also causes removal of the GluA2 subunit from the synapse via a Ca²⁺-dependent phosphorylation.81 Increased GluA2-lacking AMPARs were evidenced by increased Ca²⁺ permeability and the presence of inwardly rectifying AMPAR currents (FIG. 23B).81 Moreover, AMPAR antagonists, including NBQX (c(2,3-dihydroxy-6-nitro-7-sulfonyl-benzo[f]quinoxaline) administered within 1 hour post-seizure blocked GluA1/GluA2 post-translational events and altered function,81 suggesting that AMPAR activation was critical for these secondary ELS-induced changes. Importantly, this treatment also rescued later life cognitive and behavioral deficits.59 Similar effects on AMPARs, specifically seizure-induced GluA2 expression/function decreases, occur after KA and pilocarpine seizures. GluA2 expression/function decreases are also observed in models of autism in which seizures are common, including Tuberous Sclerosis Complex (TSC) and CDKL5 mutation disorder (CDD), where AMPAR antagonists show similar efficacy.

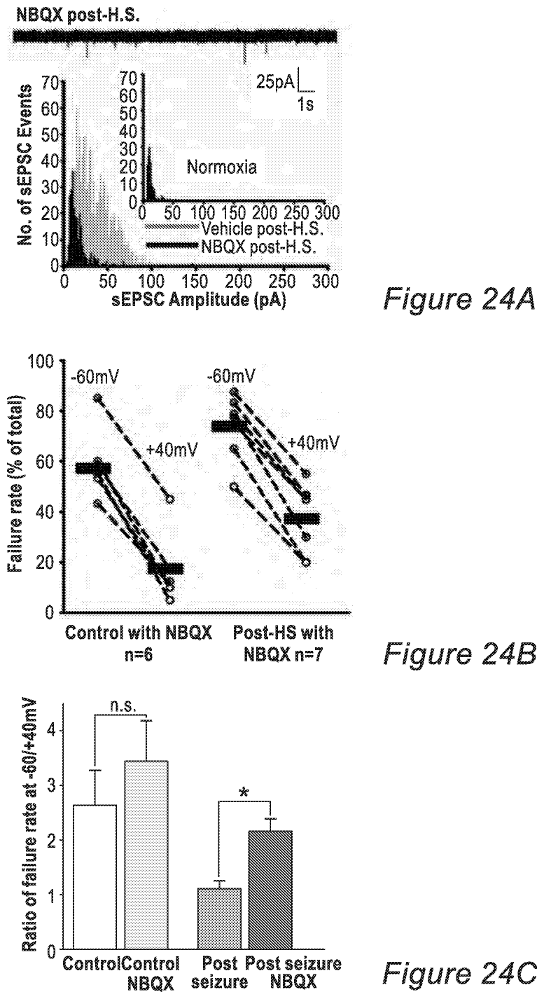

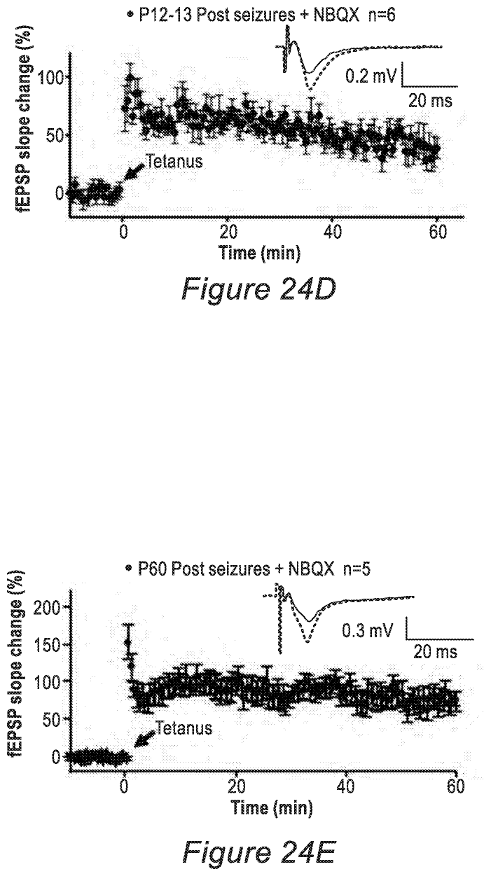

ELS-induced AMPAR dysregulation results in disrupted synaptic plasticity. Altering the synapticexpression of GluA1 levels and GluA2-lacking receptors can impact the induction of both LTP and LTD. Indeed we have shown that the increase in GluA2-lacking AMPARs from ELS is associated with both occlusion of LTP108 and a reduction in LTD.60 At any age, LTP is in part due to trafficking of GluA1-containing AMPARs to the membrane, to activate “silent” NMDAR-only synapses. Early postnatal development is characterized by an abundance of silent synapses that become “unsilenced” with coordinated activity and experience. However, ELS decrease the fraction of silent synapses (FIG. 23C), occluding the LTP of CA1 neurons (FIG. 23D,E); thus ELS prematurely convert silent to unsilent synapses due to activity dependent trafficking of receptors, impairing subsequent plasticity. Furthermore, the LTP impairment persists to adulthood, where AMPARs appear to be necessary and sufficient for the aforementioned impairments, as immediate post-seizure treatment with AMPAR antagonists such as NBQX rescues these deficits. As above, we showed that such treatment results in a rescue of 1) the GluA1 and GluA2 expression changes, 2) the increased Ca2+ influx, 3) the enhanced EPSCs (FIG. 24A) the loss of silent synapses (FIG. 24A-C) the early and long-term changes in LTP (FIG. 24D-E) and LTD. In addition, AMPAR antagonist treatment in the first 48 hrs post ELS attenuates later life social and behavioral deficits, and critical period learning deficits.

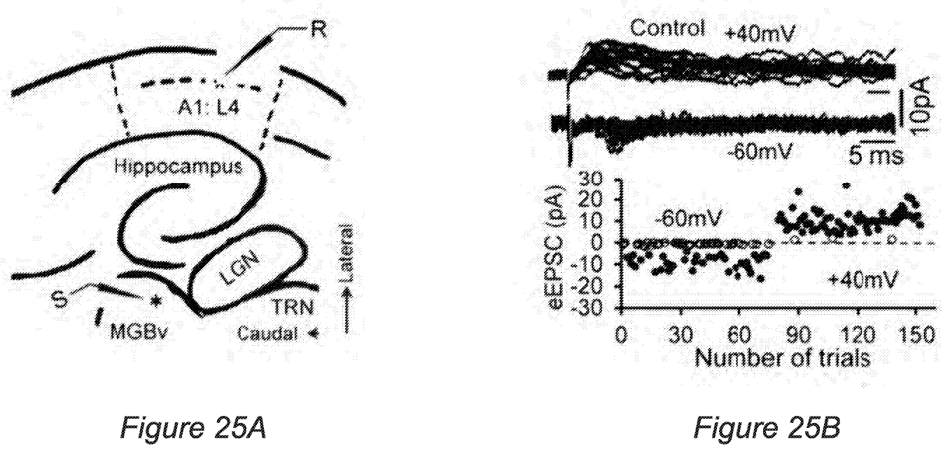

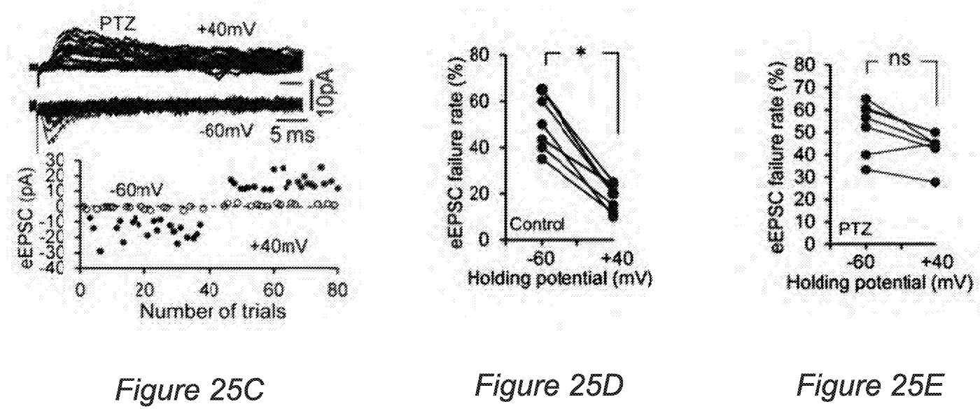

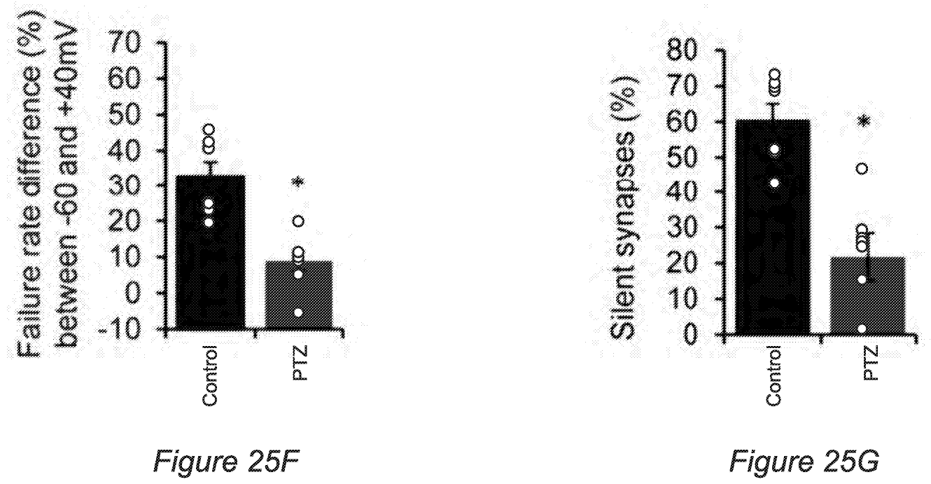

Evidence for seizure-induced altered plasticity incortical neurons. We showed that ELS induced changes in AMPARs are not unique to hippocampus, but rather a general seizure-induced mechanism that impacts other parts of the brain, as is observed in alterations to auditory cortex critical period. The auditory cortex exhibits a physiological critical period at P12-15, upon ear canal opening at P12, where tone-rearing during this window can dramatically alter the primary auditory cortex (AI) tonotopic map, as measured in thalamocortical slices. Similar to hippocampal development, the auditory critical period is characterized by a prevalence of silent NMDAR-only synapses that are unsilenced throughout this window. Induction of seizures with PTZ for 3 consecutive days (P9-11) prior to this critical period blocks tonotopic map plasticity by prematurely unsilencing NMDAR-only synapses (FIG. 25). Brief treatment with 1 dose of NBQX following seizure induction each day prevents synapse unsilencing and permits subsequent A1 plasticity. These findings reveal that ELS modify critical period regulators and that GluA2-lacking receptors may be a critical component in the neurodevelopmental impairments that follow. Given their therapeutic efficacy in several settings, we will use AMPAR antagonists as well as specific antagonists of GluA2-lacking receptors to investigate whether they also prevent ELS-induced changes in individual neuronal phenotypes.

Efficacy of specific blockers of GluA2-lacking AMPARs in models of ELS and autism. Given the potential role of Ca2+ dependent synaptic signaling pathways in functional changes we have observed, we evaluated the efficacy of IEM-1460, a selective blocker of GluA2-lacking AMPARS, both in vitro and in vivo. Given its role in synaptic plasticity and neuronal maturation in early life, we examined how the transcriptional regulator methyl CpG binding protein (MeCP2) was affected by ELS. 82 Mutations in MeCP2 lead to the autistic-like disorder Rett Syndrome and MeCP2 function can be regulated by CaMKII phosphorylation of its 5421 site. We showed that ELS increased both CAMKII and MeCP2 S421 phosphorylation, and this could be blocked by early in vivo post-seizure treatment with the broad-spectrum AMPAR antagonist NBQX or the specific GluA2-lacking AMPAR adamantane derivative, IEM-1460.82 These data suggest that GluA2-lacking AMPARs are age-specific therapeutic targets for convergent pathways in synaptic development, epilepsy and autism.

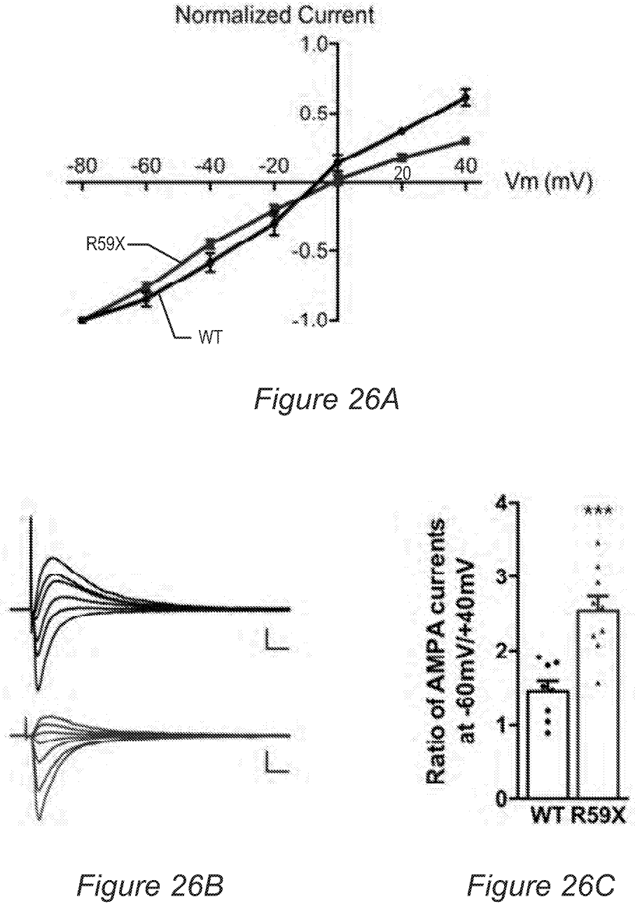

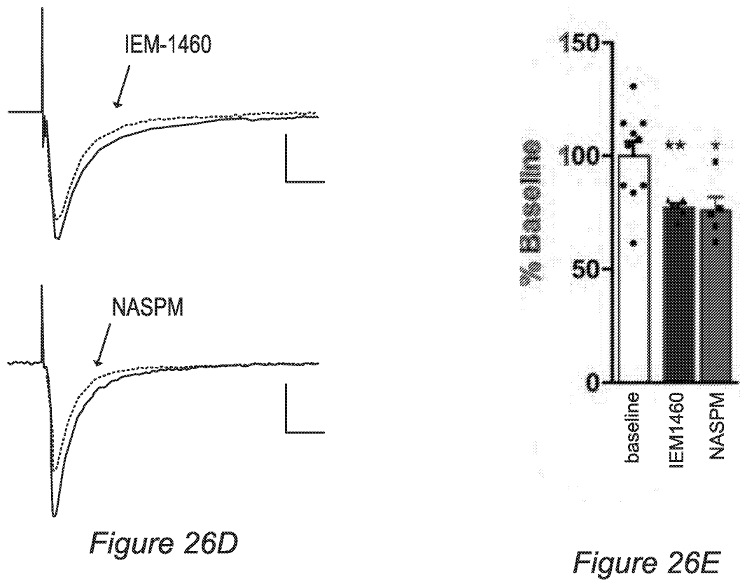

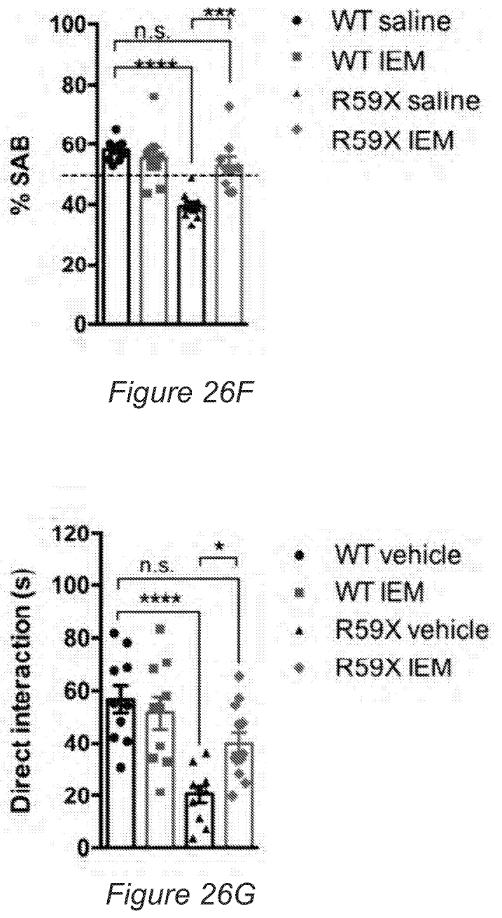

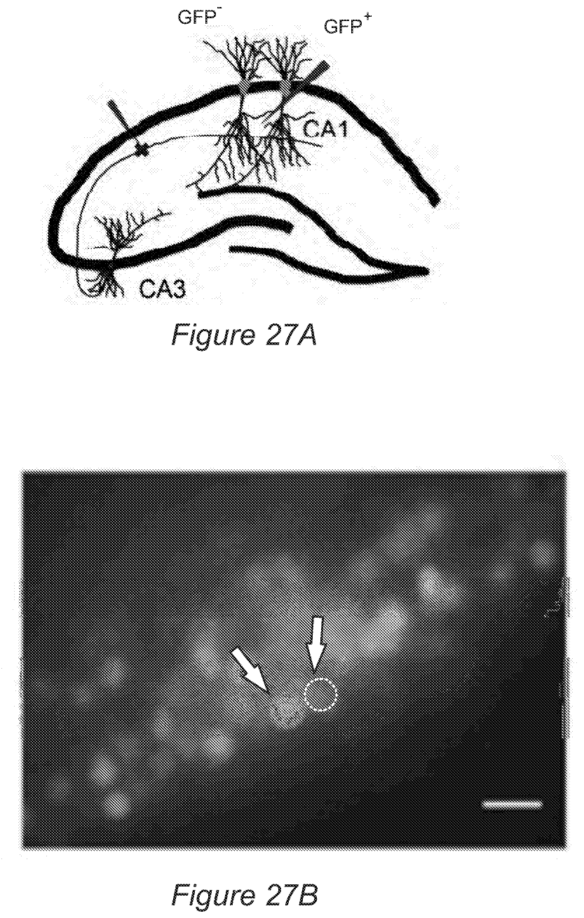

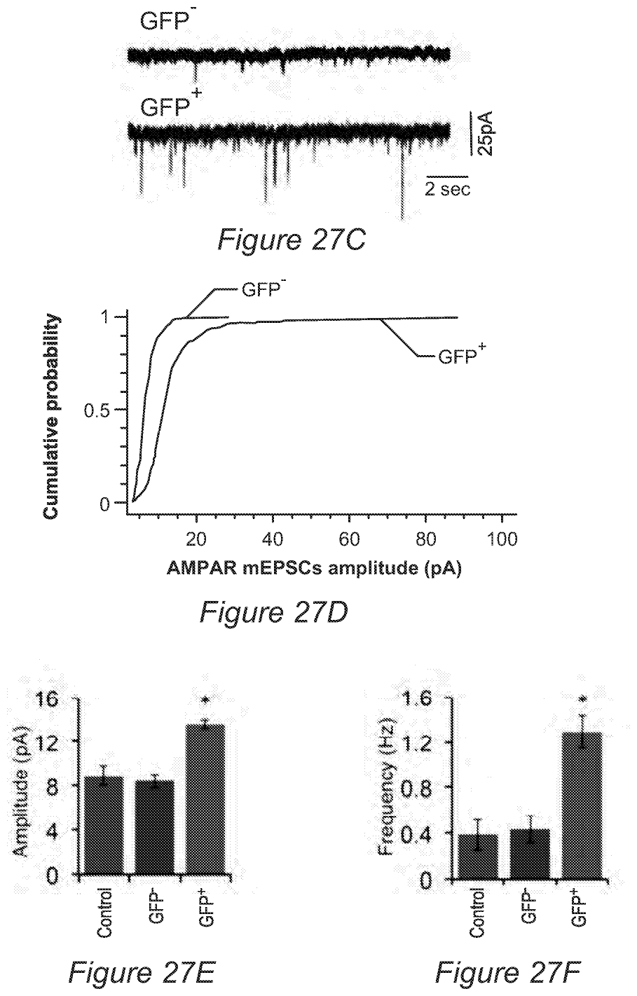

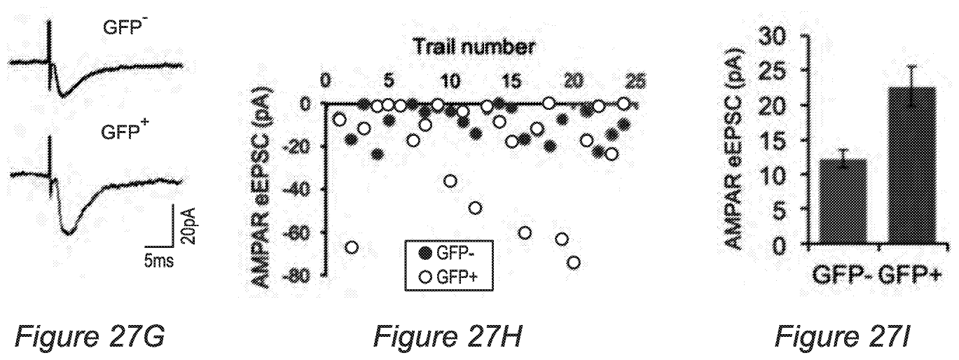

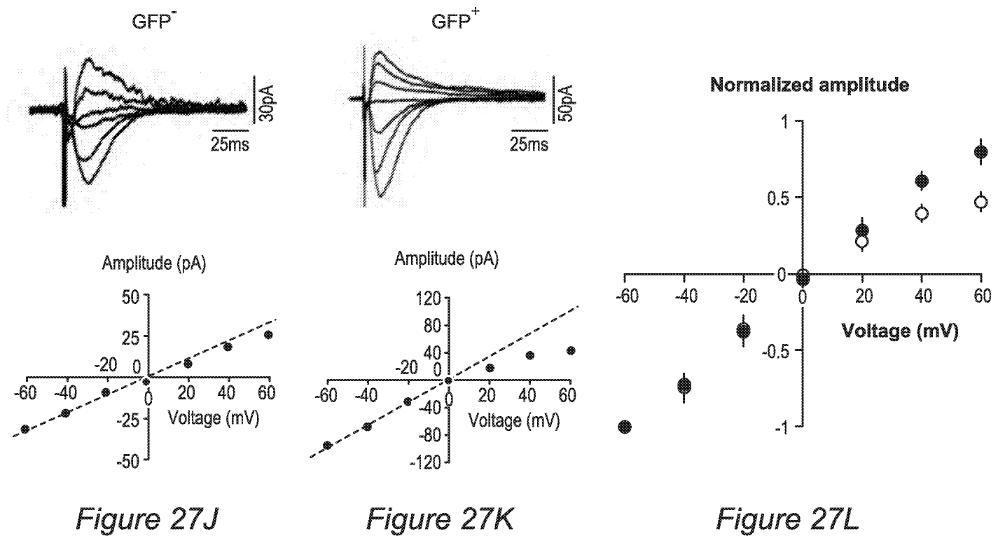

Further highlighting GluA2 dysregulation as a converging underlying mechanism across ELS, autism, and intellectual disability, we revealed a role for GluA2 dysregulation in CDKL5 deficiency disorder (CDD), a neurodevelopmental disorder characterized by epilepsy, intellectual disability and autism. The CDKL5 R59X mouse model exhibits social deficits, memory and learning impairments, as well as increased seizure susceptibility. In addition, R59X mice and human CDD hippocampal tissue exhibited a specific increase in GluA2-lacking AMPARs, accompanied in the mouse by increased rectification ratios of AMPAR EPSCs and altered LTP. Importantly, the AMPAR blocker IEM-1460 decreased AMPAR currents (FIG. 26), rescued social deficits, working memory impairments, and seizure behavior latency in R59X mice. We will thus compare the efficacy of this selective blocker versus that of the broad spectrum AMPAR antagonist NBQX in this proposal.

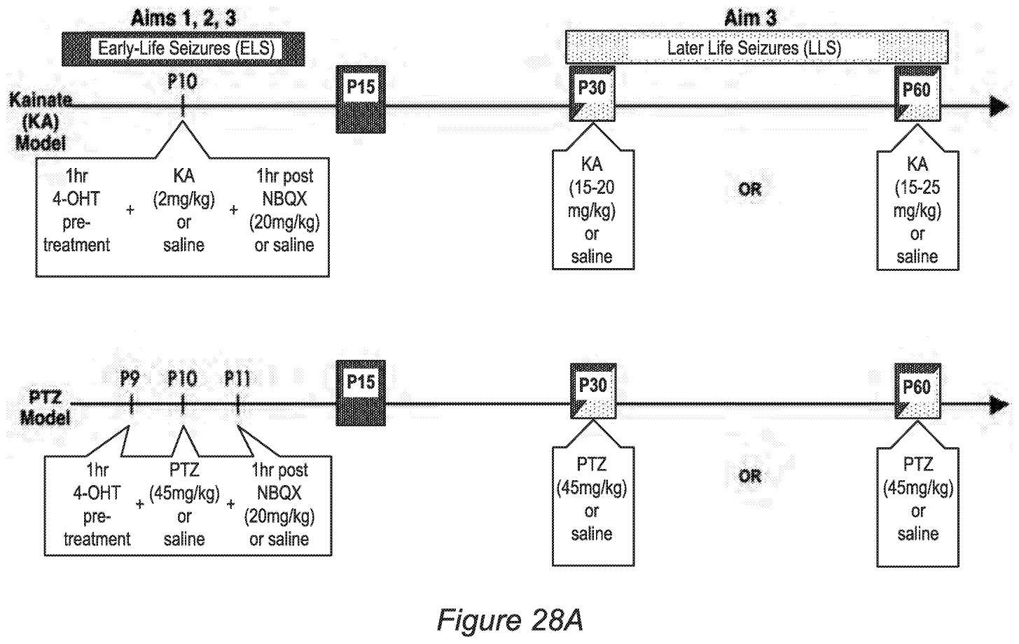

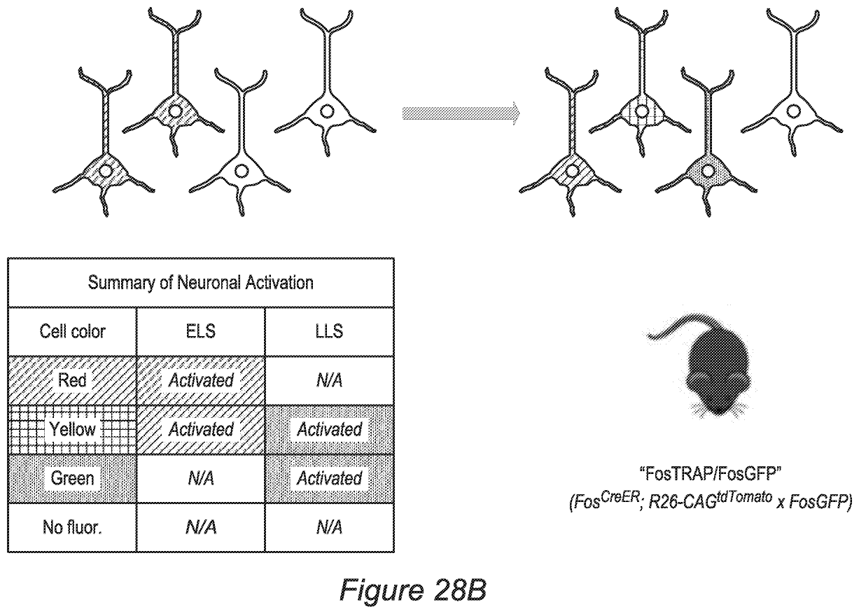

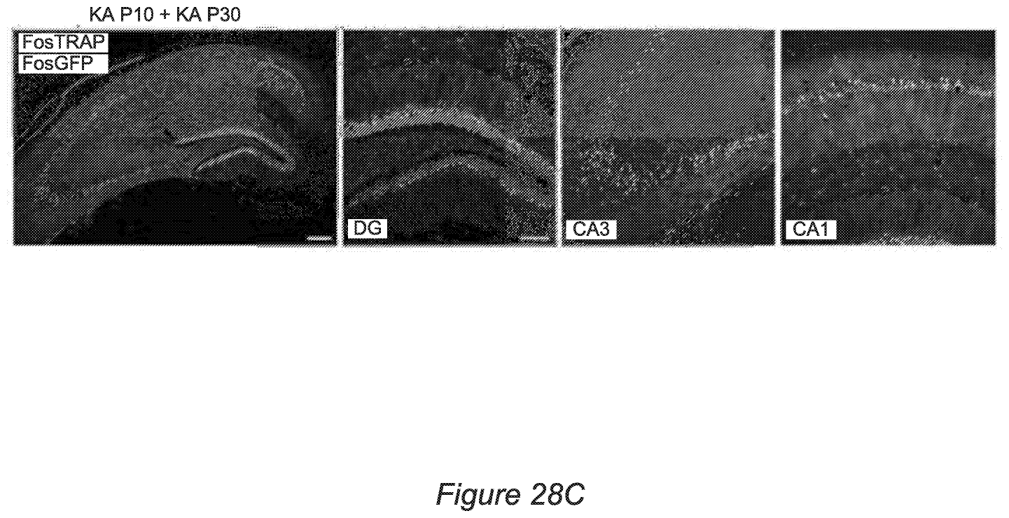

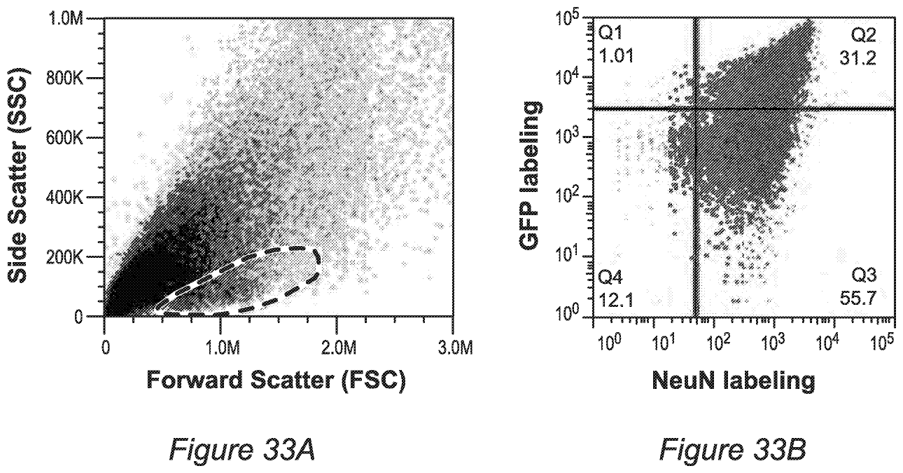

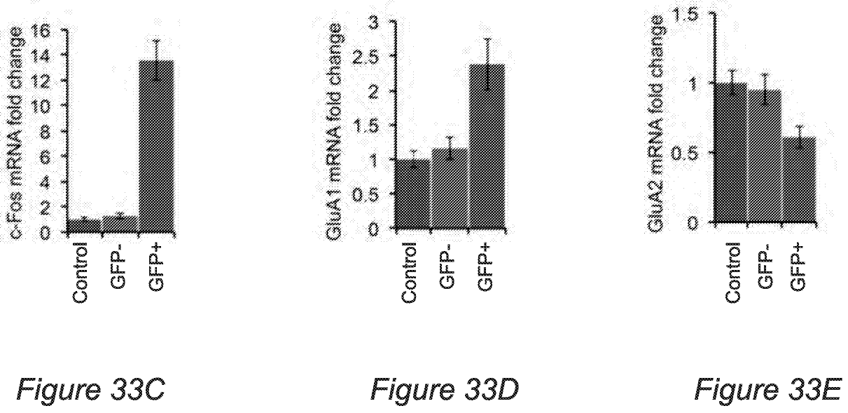

FosGFP mice show that ELS differentially activates specific neurons in CAL As above (FIG. 24) we show robust alterations in network plasticity (LTP/LTD) in adulthood, but unlike the early acute/subacute timepoints, it has been challenging to find single cell and synaptic alterations in these adult slices from the same animals that have impaired learning and memory. Hence we propose a new FosTRAP/FosGFP mouse model to unambiguously follow the subset of neurons involved in the ELS. Indeed, electrophysiological recordings in vivo and ex vivo brain slices from unanesthetized rats with spontaneous seizures and human tissue biopsies from adult patients with focal epilepsy1, 96 have shown that seizures induce heterogeneous responses around seizure onset within distinct neuronal subpopulations. In pilot experiments, we induced PTZ seizures at P10 in FosGFP mice (JAX 01435), where neurons activated by seizures can be acutely identified due to GFP from seizure-induced activation of the immediate early gene (IEG) c-fos. Strikingly, we found that ELS activate subsets of neurons (not all) in distinct neuronal populations in the hippocampus and cortex, and that these neurons undergo unique changes in AMPAR function not shared by surround cells. In hippocampal CA1, only a subset of neurons expressed GFP at 2-3 hrs post seizure (FIG. 28A-B), and these GFP+ neurons were highly enriched for rapid increases in postsynaptic AMPAR function characteristic of our earlier work: significantly enhanced AMPAR mEPSCs amplitude and frequency (FIG. 28C-F), increased amplitude of minimally-evoked EPSCs (FIG. 28G-I) and inwardly rectifying AMPAR currents (FIG. 28J-L). In addition, GFP+ cells exhibited diminished NMDAR-only silent synapses (FIG. 33A-F), and increased AMPAR single-channel conductance (FIG. 33G-J), which was not observed in surround GFP− cells or those from control no seizure littermates.

Such findings highlight that only a subset of neurons undergoes the signature physiological dysfunction immediately following ELS. However, as FosGFP fusion protein only persists for 4-8 hrs, it is not possible to determine whether the same neuronal subpopulation that drives impaired network plasticity acutely is also the population that is involved in the long-lasting behavioral changes associated with ELS. While novel, a limitation of this FosGFP work and other prior ELS work has been the lack of ability to track the evolution of changes to single neurons throughout animal's lifespan. To address this, we created a new transgenic mouse model.

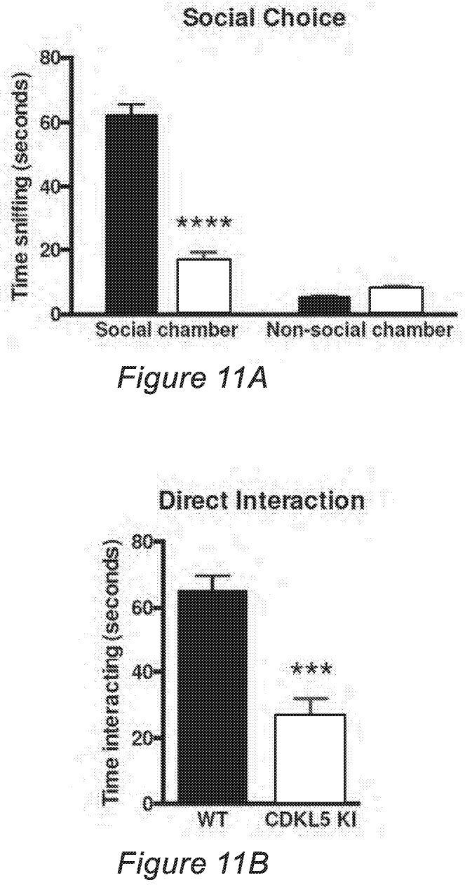

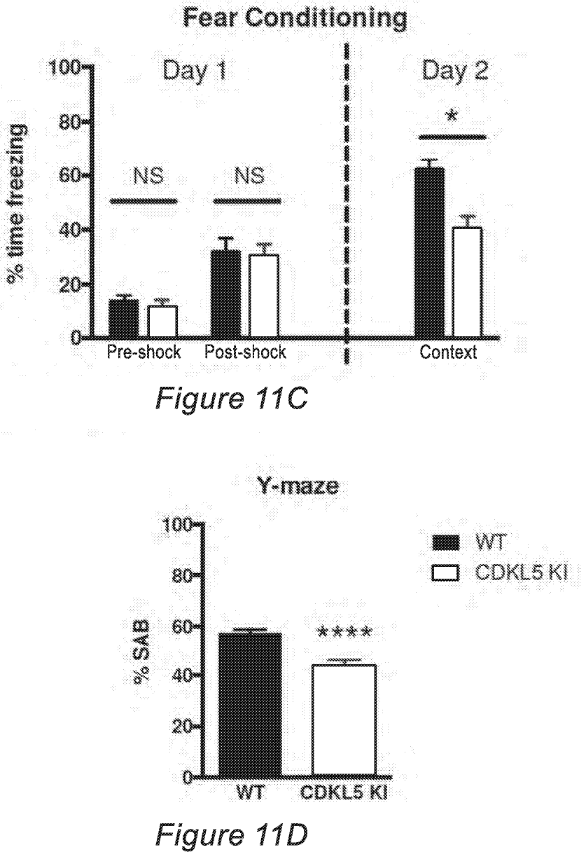

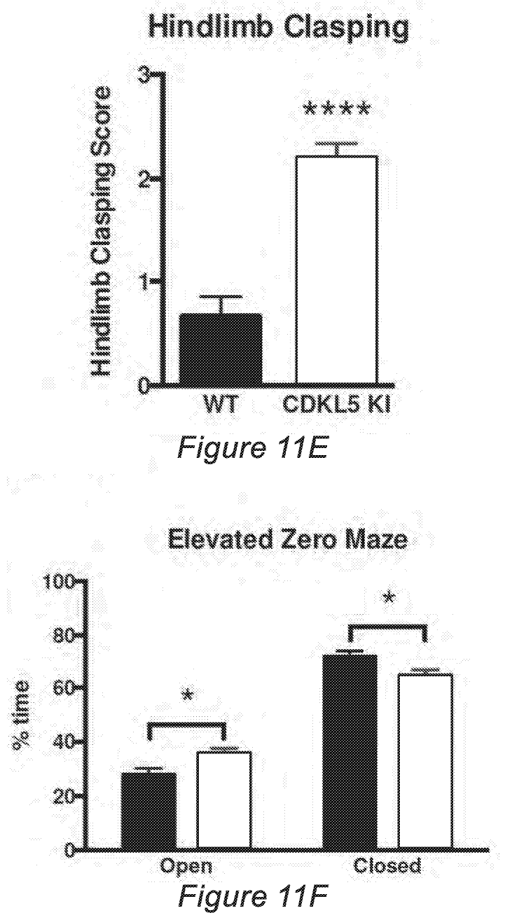

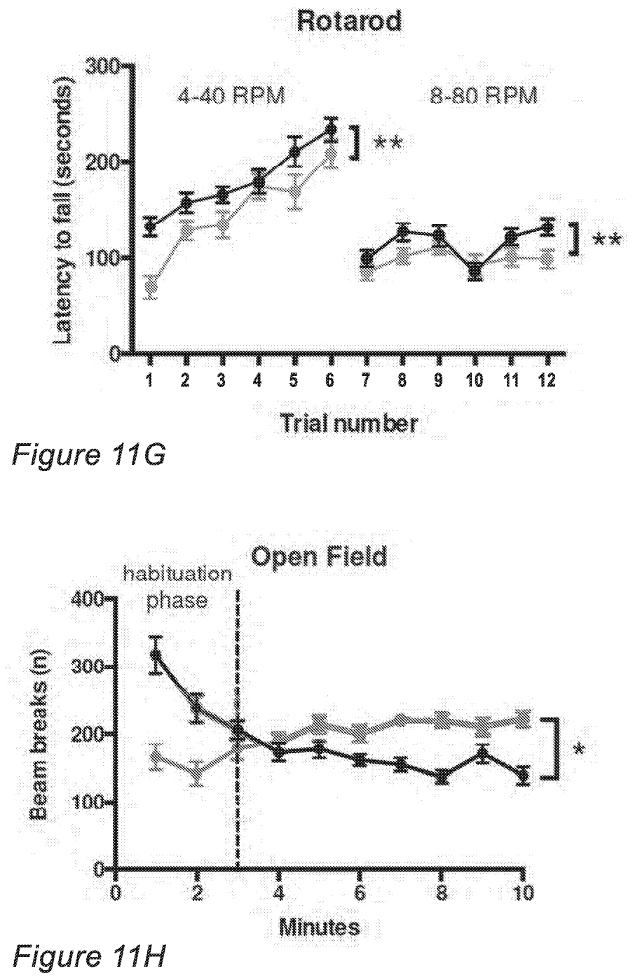

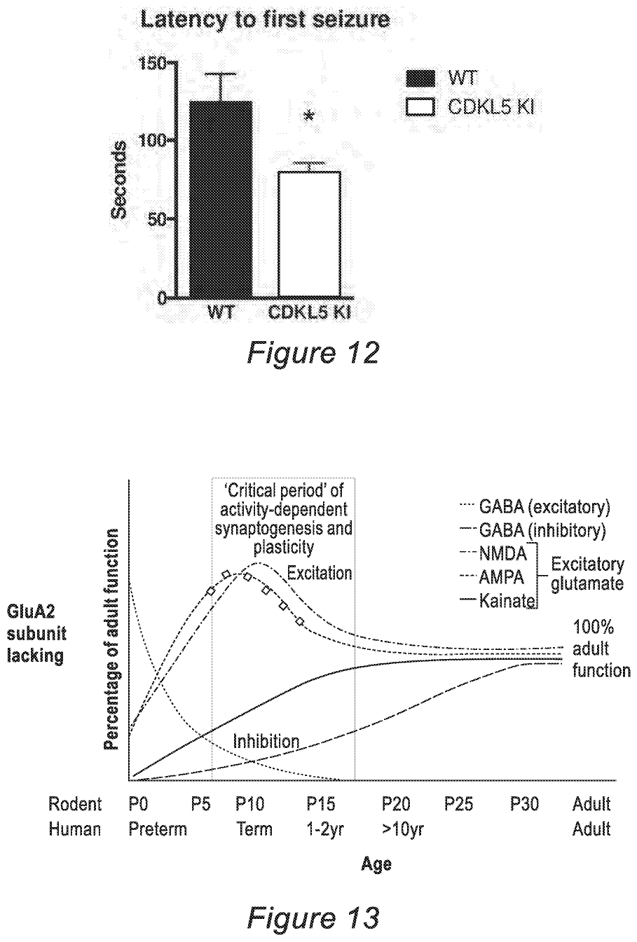

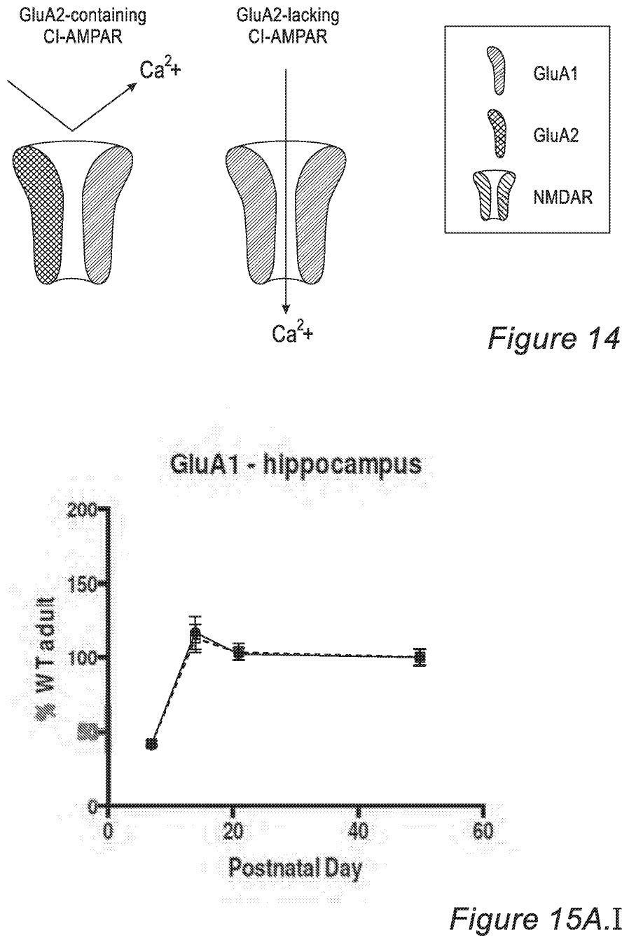

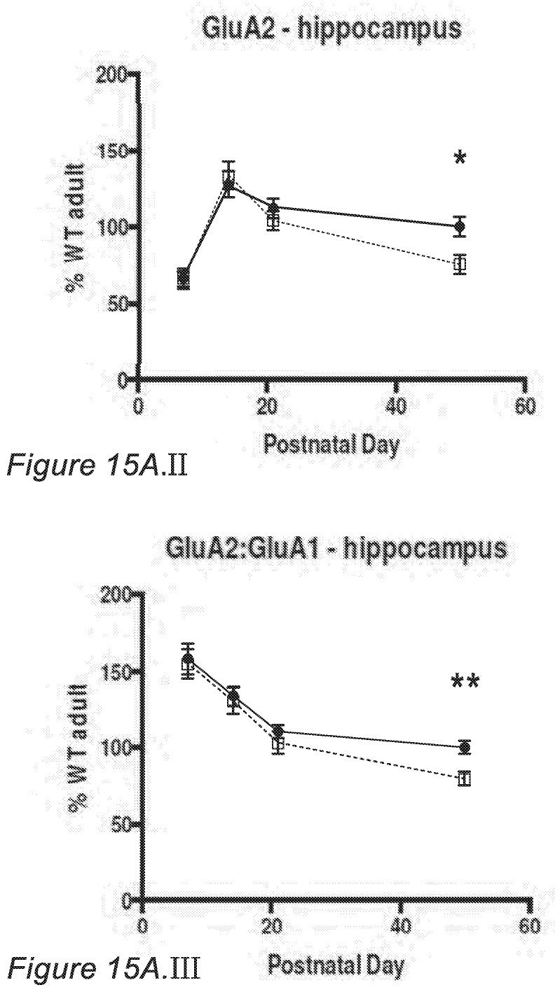

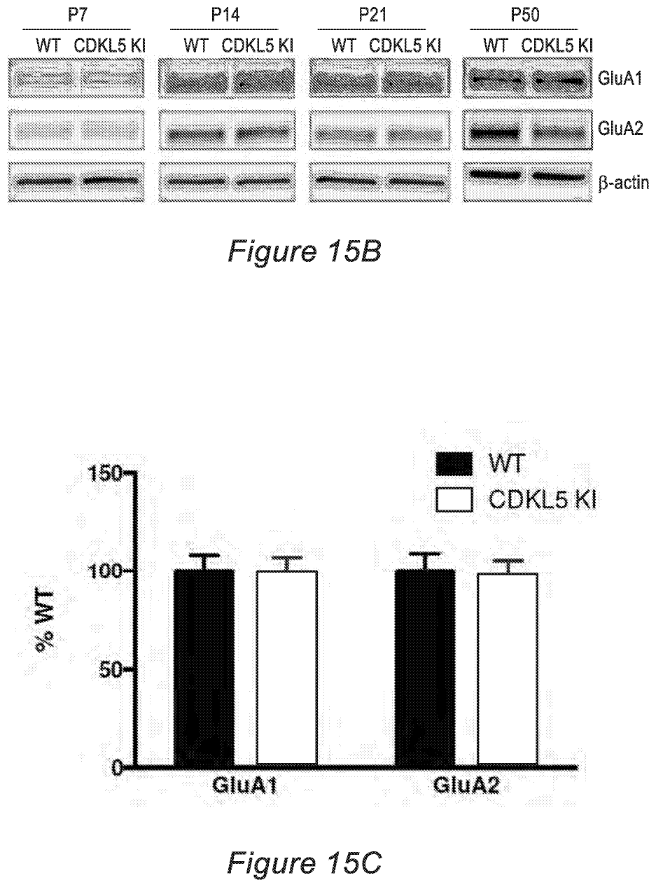

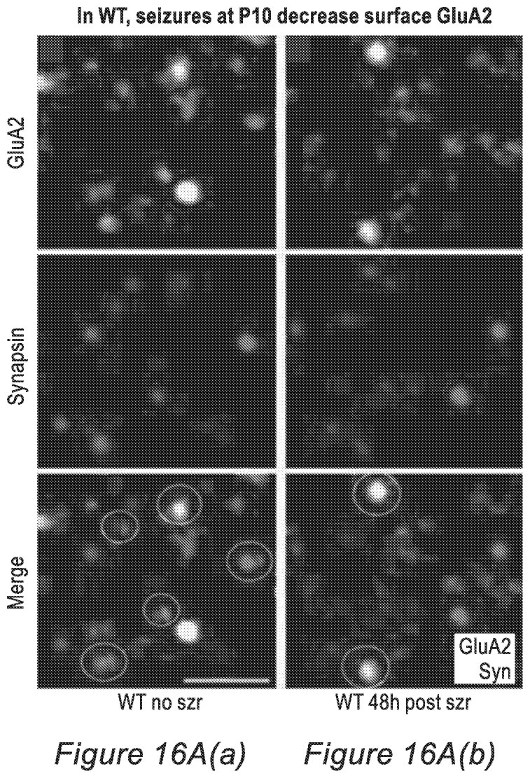

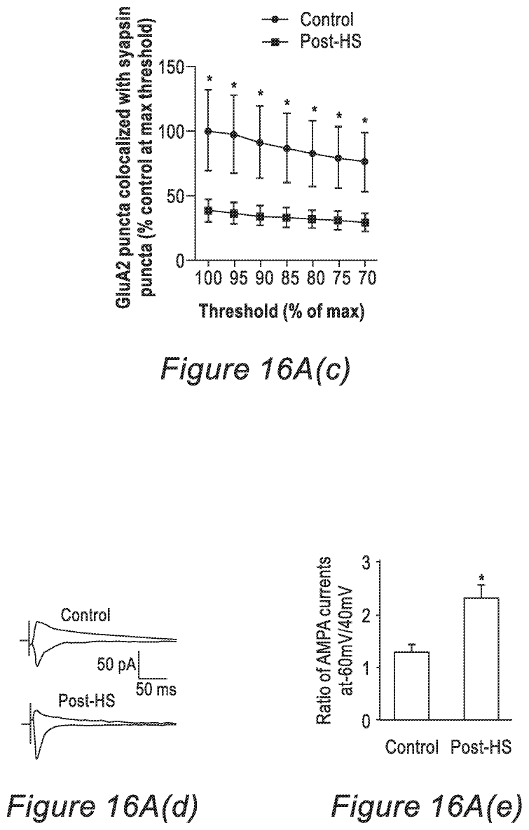

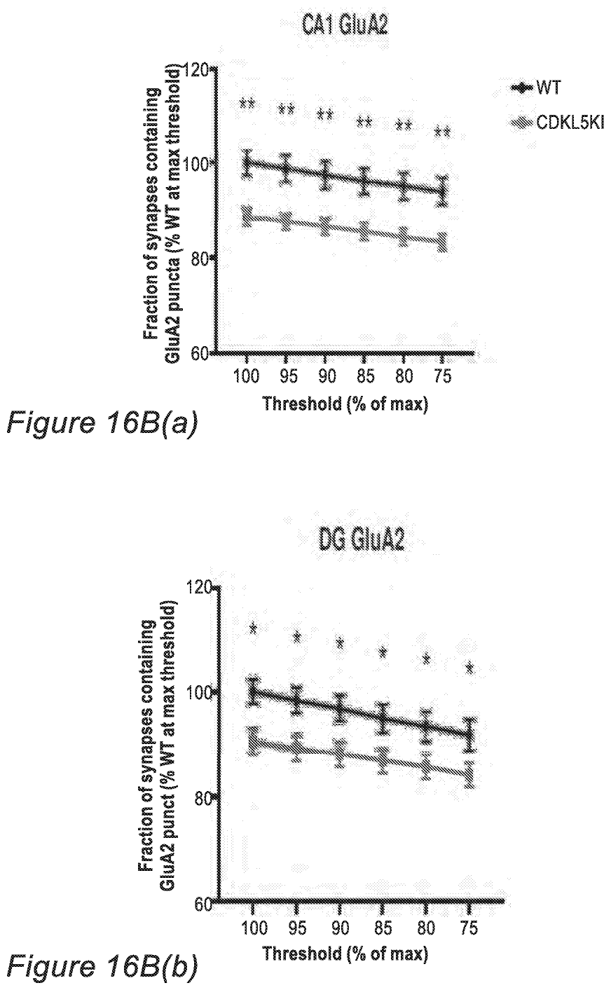

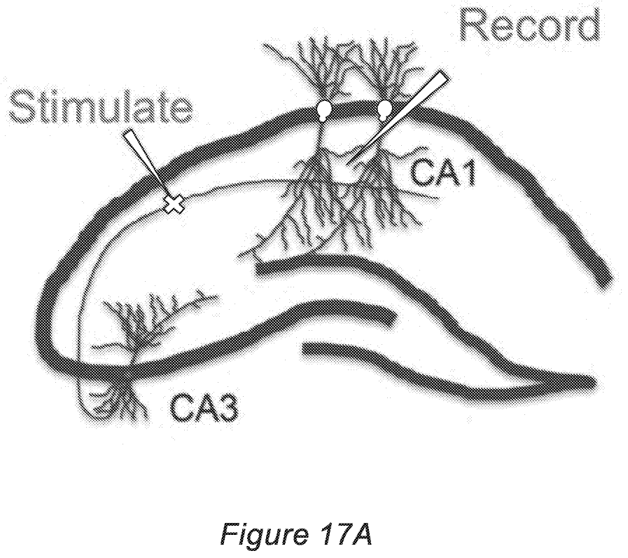

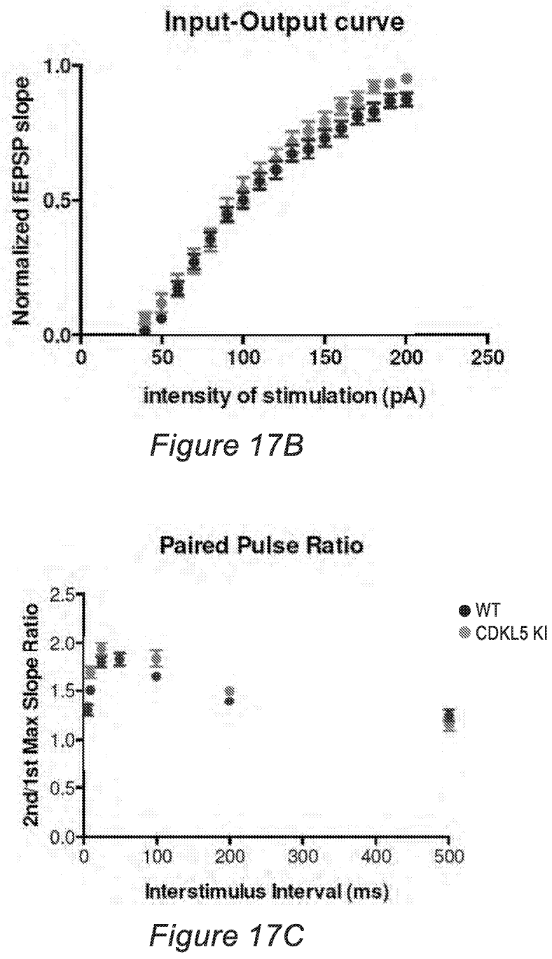

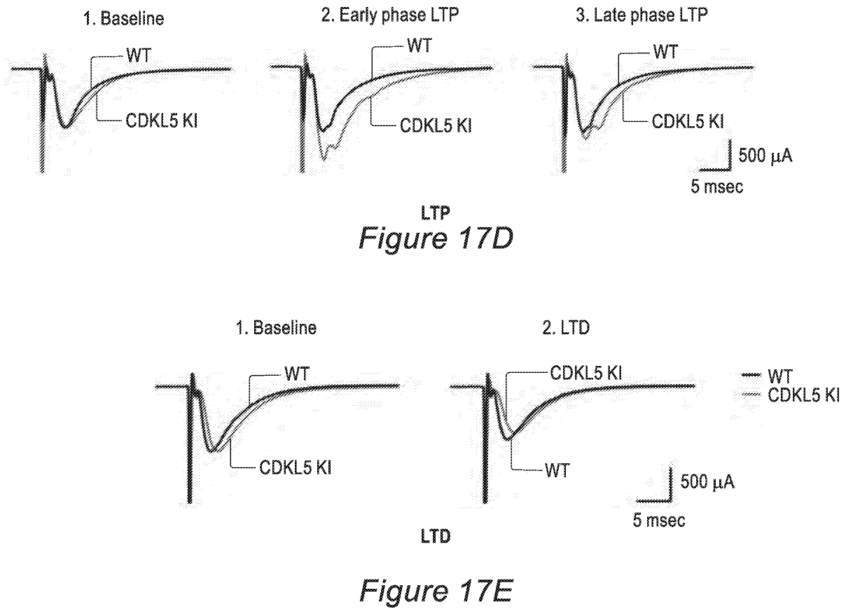

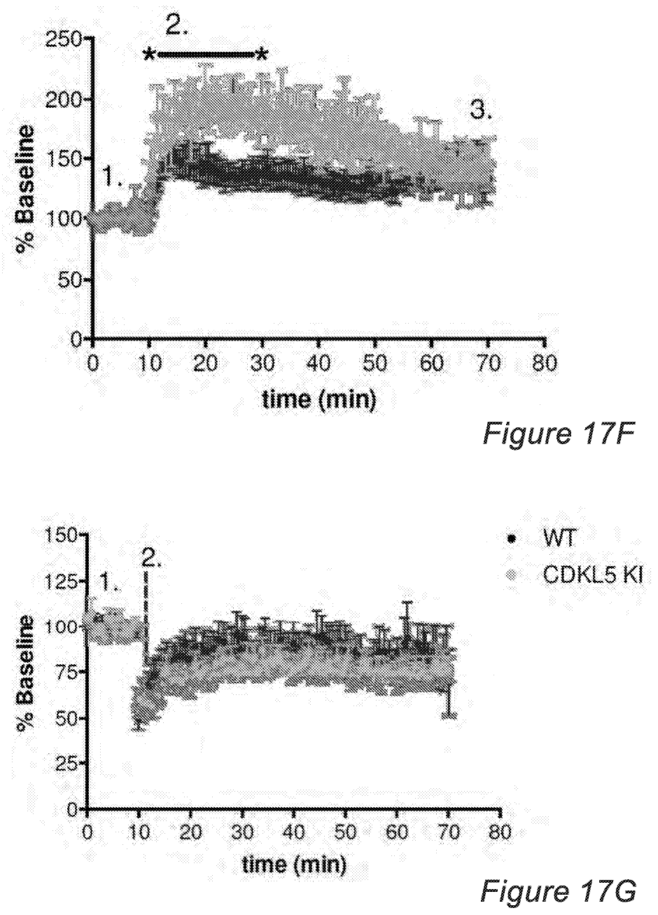

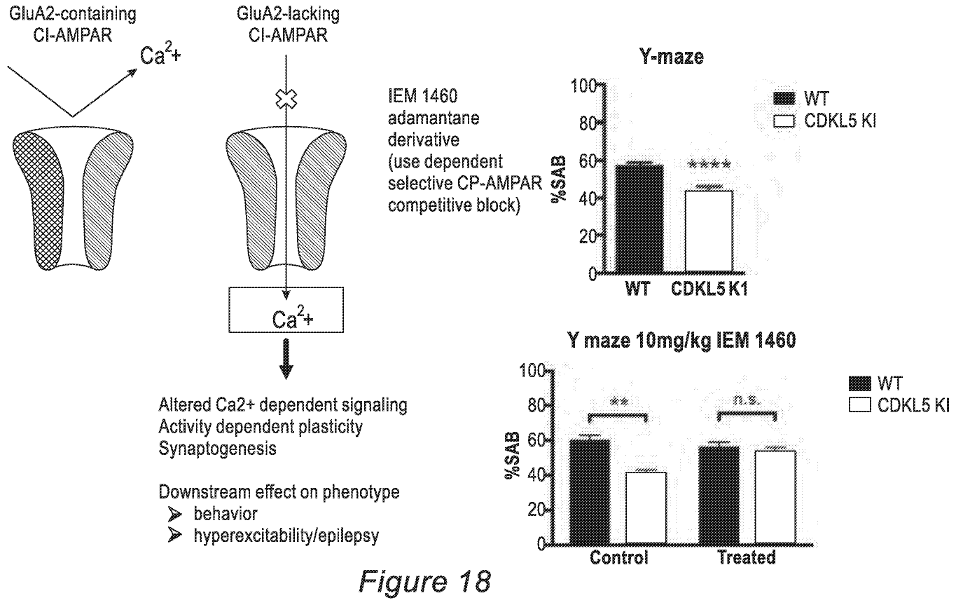

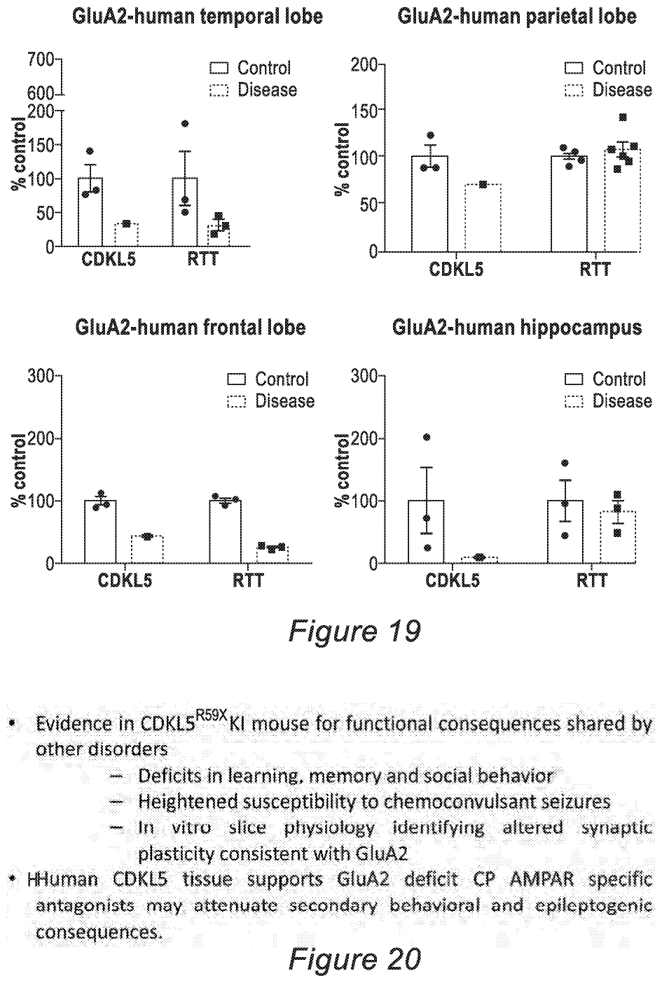

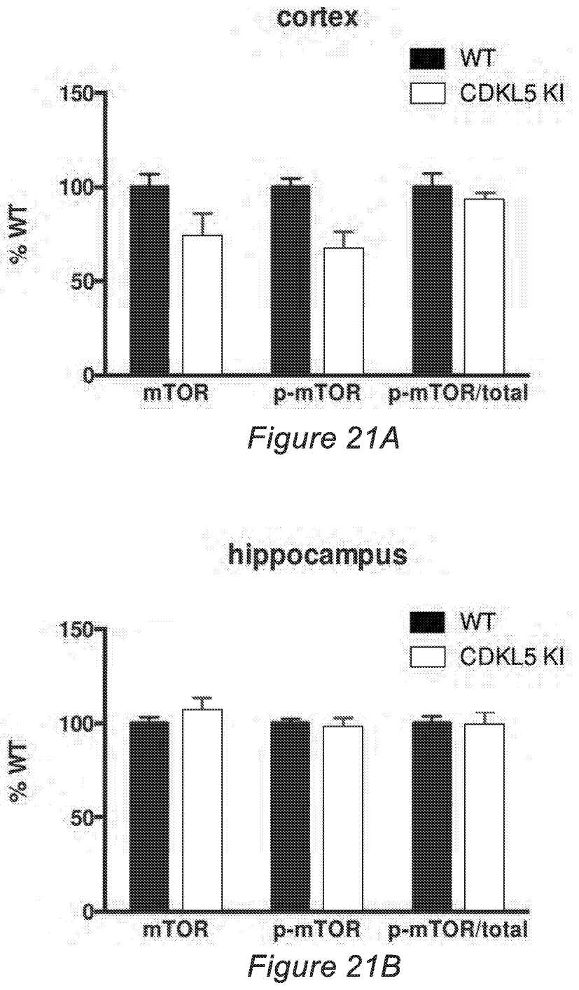

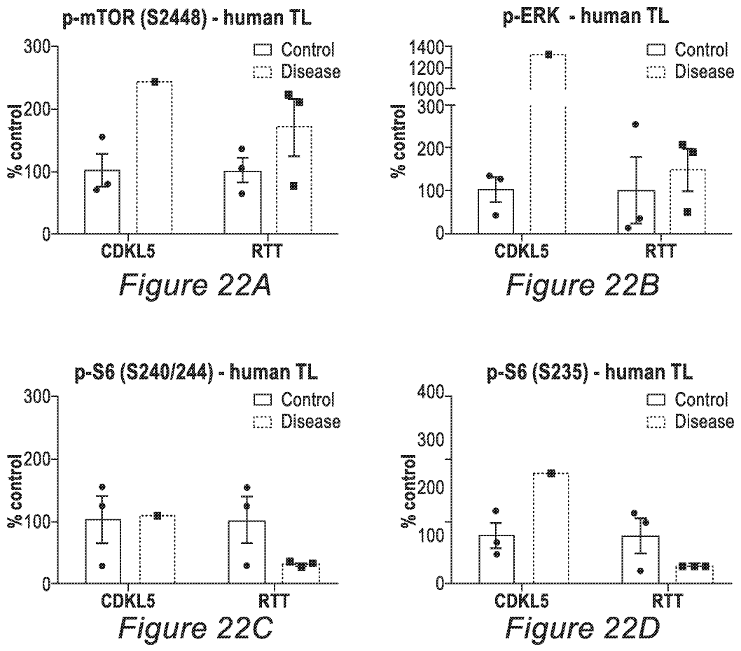

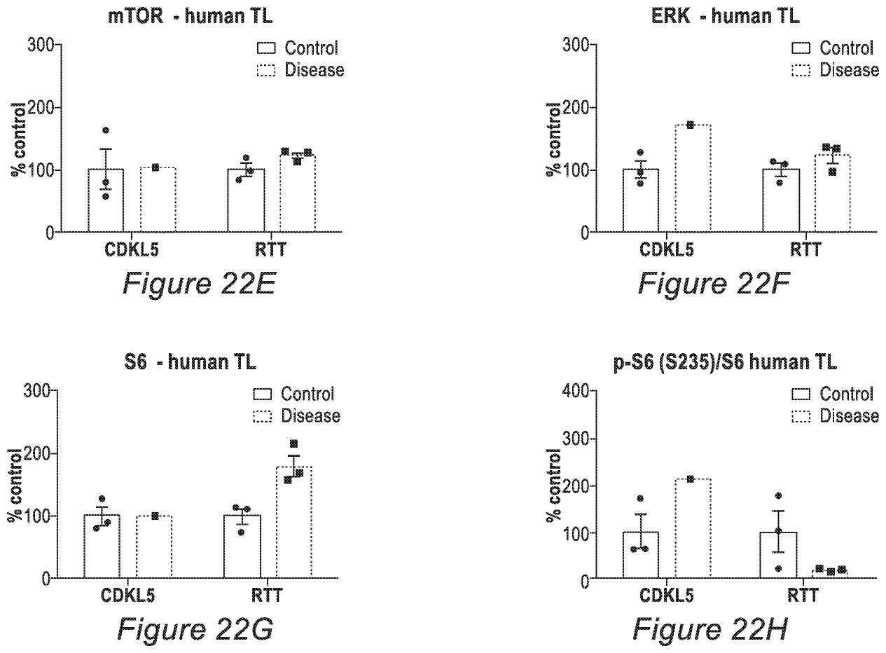

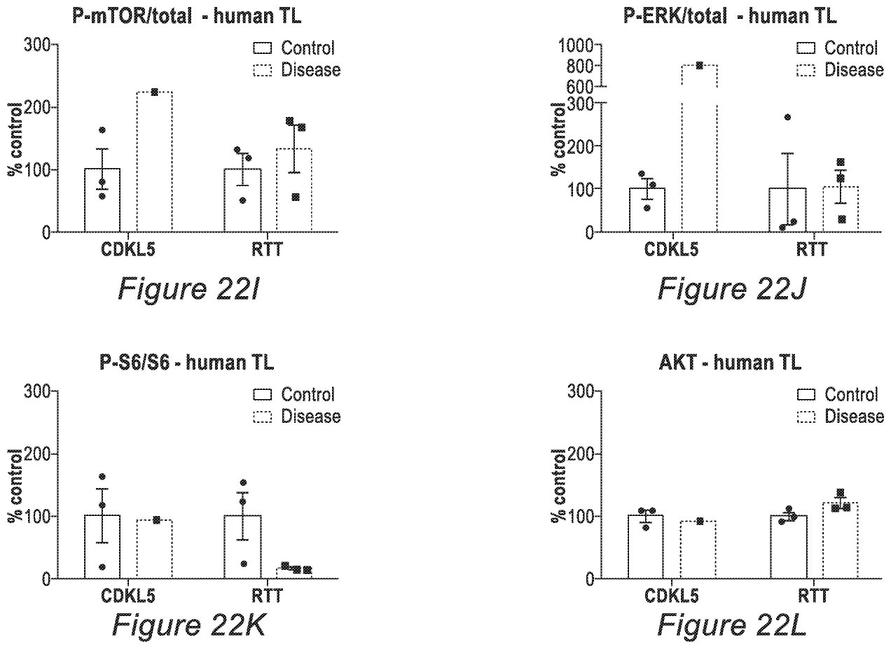

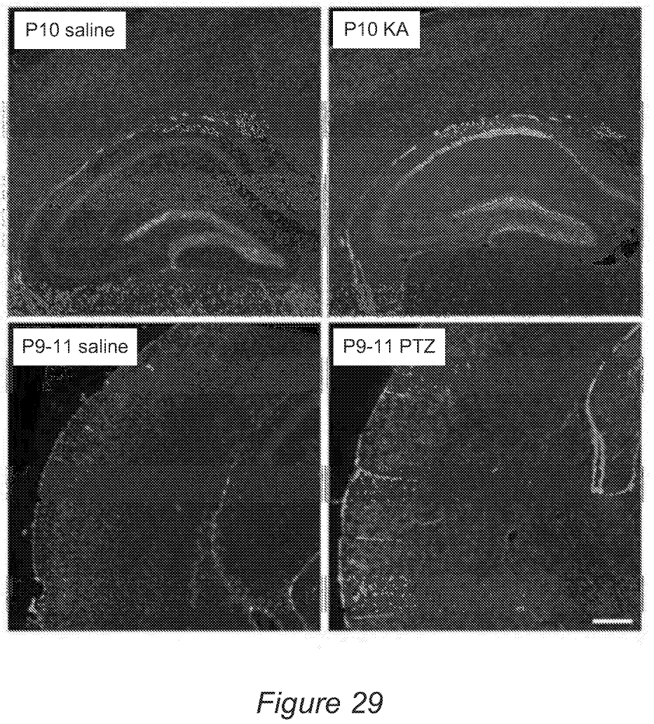

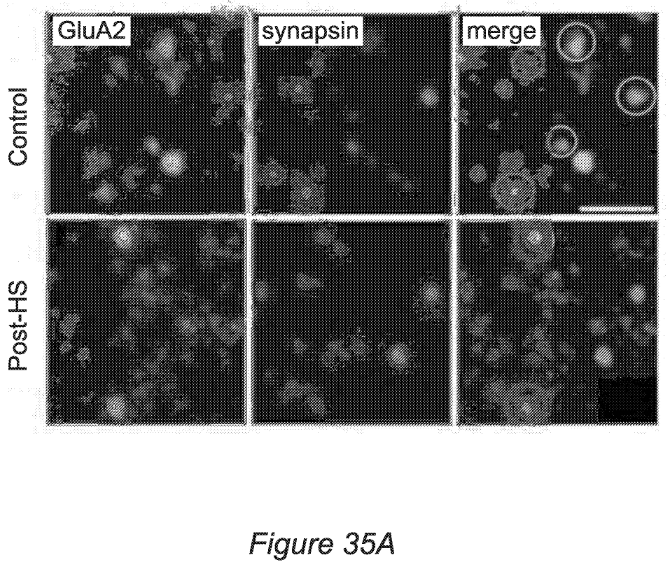

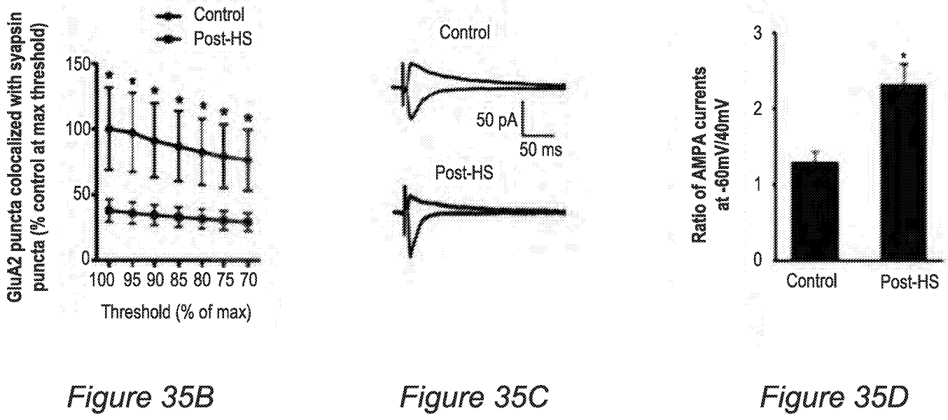

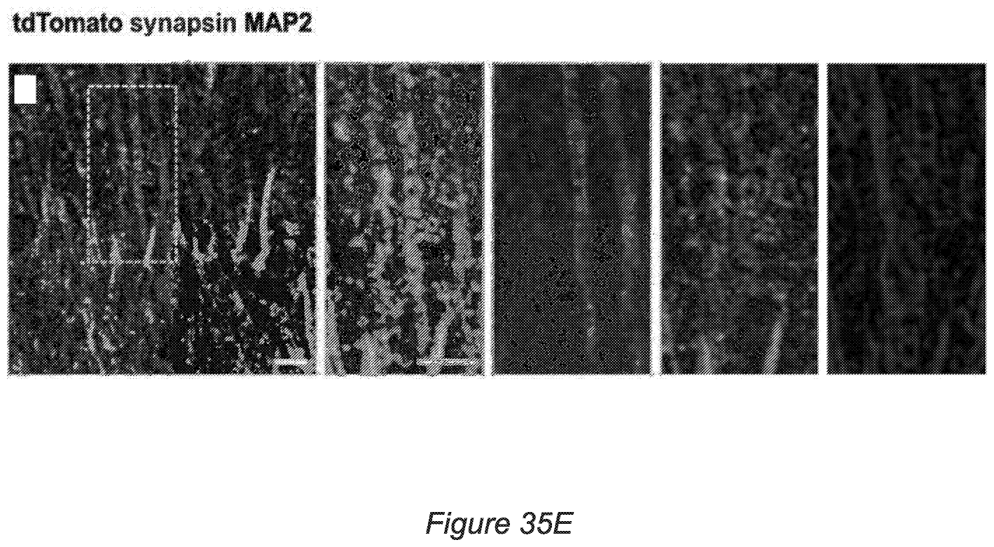

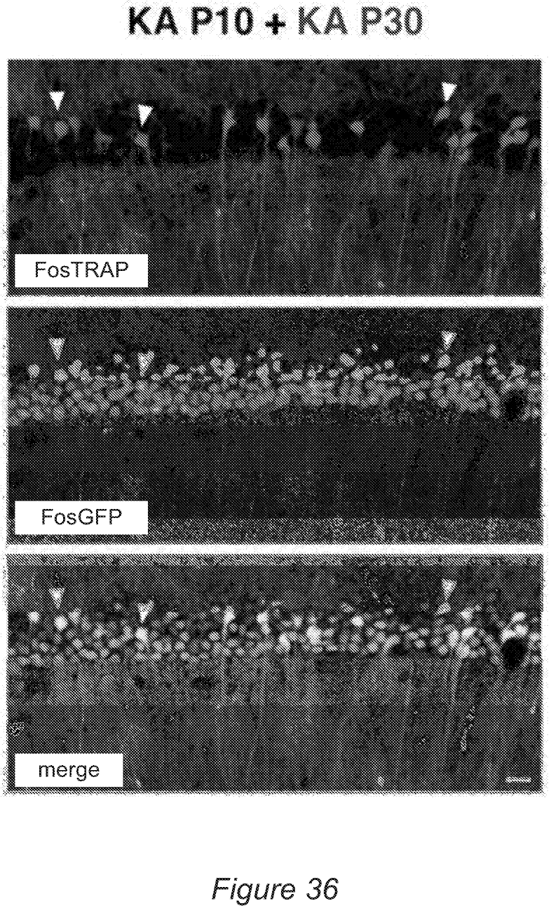

Adapting the FosGFP mouse to study the long-term effects of ELS by generating a new FosTRAP/FosGFP model to track changes in synaptic plasticity across the lifespan. Given that our results with the FosGFP revealed that only a subset of neurons responded to ELS, we wanted a model to be able to track these changes lifelong. In order to do this, we have adapted the FosTRAP mice (targeted recombination in active populations (TRAP)), described by Guenthner et al, that allows permanent labeling of neurons undergoing c-fos activation with the tdTomato (tdTom) fluorescence marker during a specific time window when an additional effector (4-hydroxytamoxifen, 4-OHT) is administered to the animal. Our adaptation was to cross this mouse with the aforementioned FosGFP mice to generate a novel “FosTRAP/FosGFP” mouse that utilizes both permanent and acute/transient labeling of active cell populations driven by the IEG Fos to compare neuronal subpopulations that were activated to specific identifiable events. Hence our new mouse line allows permanent labelling of cells using the TRAP tdTom method, and allows us to also reactivate c-fos induction with a later life seizure (LLS) in order to identify cells where activation occurred exclusively in the first seizure (red) or both (yellow) (FIG. 29). The FosTRAP component is useful to permanently label neurons that are activated in a distinct time window, as the fluorescence tag is conditionally expressed in neurons when both the c-fos activating seizure and 4-OHT, a tamoxifen/estrogen mimetic, are present together (FIG. 29).33, 103 We adjusted the time course of both stimuli and 4-OHT administration so that it persists for 4-6 hrs to maximize specific “TRAPing” of neurons only related to the ELS.19, 103 The result is a selective and permanent cytoplasmic and nuclear expression of tdTom fluorescent protein to “TRAP” (tag) neurons activated by ELS. As we have crossed this mouse with the FosGFP transgenic line,7 we can also take advantage of IEG expression and fluorescent tags. This eGFP fluorescence only lasts in the cell for 4-8 hrs and can thus be used to label neurons activated within hrs prior to harvesting the tissue. This novel FosTRAP/FosGFP mouse line allows for the identification of neurons originally activated by ELS and enables us to follow them over extended periods of time with in vitro electrophysiological investigation (FIGS. 28, 31, 32), RNA transcriptomics by fluorescence-activated cell sorting (FACS) (FIG. 11) and lightening terminator-transcriptome in situ analysis (LT-TISA) (FIG. 35), as well as protein expression by immunohistochemistry (IHC) and confocal microscopy (FIGS. 36, 37).

1. New transgenic mouse. We spent over 12 months breeding transgenic lines together to create a new mouse that allows for labeling of neurons at 2 distinct points of c-fos activation. These mice can be used to label cells activated by numerous triggers including experience, environmental changes, learning, injury, seizures, addicting stimuli, and pharmacologic treatments.

2. Conceptual novelty. Conceptually this example will be the first to examine the lifespan effect of ELS on specific neurons, rather than by blind patching or network level observation. The application of fluorescence-guided whole cell patch clamp investigation in this setting is novel.

3. Application of FACS for seizure-induced neuronal subpopulations. The ability to label cells differentially allows for the use of FACS, which is novel in the setting of examining seizure-induced changes in specific transcriptomes in differentially activated subpopulations of neurons. This technique could be expanded to many other investigations using the FosTRAP/FosGFP mice.

4. Application of LT-TISA method for dendritic RNA analysis. We will employ novel transcriptome in situ analysis (TISA) approach, which to date has not been utilized in TRAP-labelled cells to specifically analyze single cell body and single dendritic RNA. This technique has never been used in an animal model of ELS and the approach of targeting neurons labelled by ELS at different intervals post-seizure is novel, and will complement the nuclear transcriptome analysis from FACS.

5. IEM 1460 as novel therapy. We will employ IEM-1460, a selective GluA2-lacking receptor blocker that has not been used in vivo in either model, as a novel strategy to target this critical AMPAR subset in seizure-induced dysplasticity. If successful, this justifies development of a specific GluA2-lacking receptor antagonists for human use, as only broader spectrum AMPARs are available that have unwanted side effects like sedation and decreased cognition. Targeting GluA2-lacking receptors in a use-dependent way may be a superior approach.

Approach:

It is clear from our preliminary data and others that seizures elicit a heterogeneous activation pattern in both immature and adult neuronal networks. Tracking alterations over time of synaptic glutamate receptors in neurons activated by ELS over time is a specific challenge given that they occur in the midst of the synaptic critical period, including the refinement of synaptic connections and the dispersion of neurons with development, which makes it difficult to localize neurons for functional studies later in life despite the persistence of impaired synaptic plasticity and cognitive deficits. Similarly, sampling of a neuronal population for gene and protein expression may fail to show alterations occurring in a small, critical, subset of cells. Here we will use FosTRAP/FosGFP mice first to permanently label cells that are activated by ELS, follow these cells over time for changes in synaptic function, plasticity and related protein and gene expression. We will focus on two brain regions (hippocampus and auditory cortex), where we have shown that early post-seizure treatment with AMPAR antagonists rescues later synaptic plasticity and behavioral deficits. We will use this proven treatment paradigm as a tool to test our hypotheses that there are distinct treatment-induced changes in AMPAR expression and function in ELS activated neurons versus neurons in control animals without ELS and if treatment attenuates those changes, it serves as proof of their critical contribution to long-term deficits after ELS. Finally, we can use FosTRAP/FosGFP mice to test our hypotheses that cells activated by the first seizure are preferentially re-activated by a second seizure later in life, indicating a permanent decrease in threshold for excitability, consistent with enhanced AMPAR function. Improved understanding of these specific neuronal populations will assist in future studies of other mechanisms of seizure-induced modification of networks during development and later adulthood beyond the scope of this proposal.

We will focus on AMPARs, fully aware that all seizures are multifactorial, but because previous work implicates AMPARs, and possibly GluA2-lacking AMPARs, as necessary and sufficient for the cellular alterations, we will be primarily assessing specific aspects of their function as outcome measures. We are specifically examining the synapse given our robust finding in multiple models that AMPAR antagonism is both antiepileptogenic and protective against cognitive deficits and impaired plasticity. The synapse is a convergence point for the likely many upstream derangements in network function, and therefore an ideal target of study.

All groups ID and data sets will be coded to blind researchers during data acquisition and analysis. The mice will be maintained on C57BL/6J background, and new founder mice will be purchased from Jackson labs regularly to prevent genotype drift. Both sexes will be used throughout and sex will be tracked and analyzed for differences in all data sets, as some ELS studies suggest there may be a gender effect with males showing greater susceptibility to ELS long term effects than females. To assure reproducibility and test generalizability of our hypotheses, we will employ 2 well-established ELS models: KA (a primarily limbic seizure model) and PTZ (primarily thalamocortical seizure model), to study neurons in hippocampus and cortex, respectively. We will only enroll mice in our study that have reached stage 5 seizures.

Importantly, neither seizure model induces neuronal death in early life. In the rat, KA-ELS results in later life abnormal working memory, fear conditioning, socialization, and increased anxiety. Seizures are not observed outside of this window or in adulthood9 although seizure thresholds are lowered.50 Adult rats exposed to prior KA-ELS exhibit impairment in synaptic plasticity and hippocampal dependent memory17 and GluA2 downregulation, similar to those we have reported after hypoxia-induced ELS. We and others have reported PTZ-induced ELS induce long term network hyperexcitability, and even decreases in the adult seizure threshold. Unlike KA and PTZ, hypoxia does not induce consistent seizures in the mouse, and here we will use the KA and PTZ as we found them more reliable in the mouse.89 While the KA-ELS model has been widely used, the PTZ-induced ELS model has more been found to impair critical period plasticity (auditory cortex with failure to tone rear), as well cause selective upregulation of AMPAR EPSCs, loss of silent synapses and decreased synaptic plasticity. All these effects are reversed by NBQX post-treatment, suggesting a critical role for AMPARs in the genesis of these long term changes. General Methods: Seizures will be induced using 2 protocols: either by KA for examination of hippocampal CA1 neurons or by PTZ for examination of auditory cortical neurons. For some experiments, mice will only get ELS and no subsequent seizure induction (FIG. 29), while in others the mice will be subjected to a second later life seizure (LLS) at either P30 or P60. All experiments will be performed on genotype confirmed FosTRAP/FosGFP mice. We confirmed in these mice with IHC that c-Fos expression after seizures does not differ from the pattern in wild type mice: there is no signal attenuation in number or pattern of c-Fos+ cells by IHC in these mice, similar to what is reported for the FosTRAP mice.33 4-OHT results in about 6 hrs of induction, so mice will be pre-treated with 4-OHT (5 mg/kg for P10 mice) 1 hour prior to ELS to obtain coverage for the established activation of c-fos transcription/translation following a single seizure, and thus triggering CreERT2-mediated expression of tdTom in active cell populations during ELS.28 Furthermore, tdTom expression reaches steady state/maximal by 72 hrs, so all mice will be studied at time points after 72-hour. All FosTRAP/FosGFP mice used as no seizure controls will also receive 4-OHT injection at P10 for KA and P9-11 for PTZ.

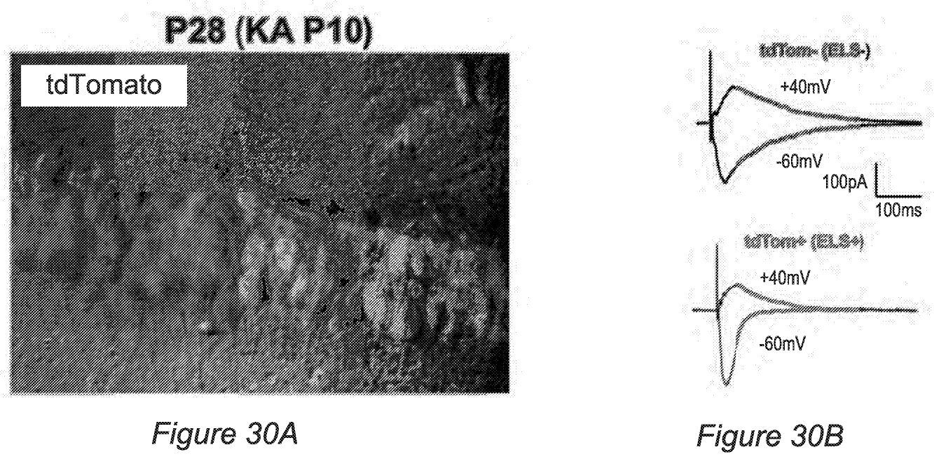

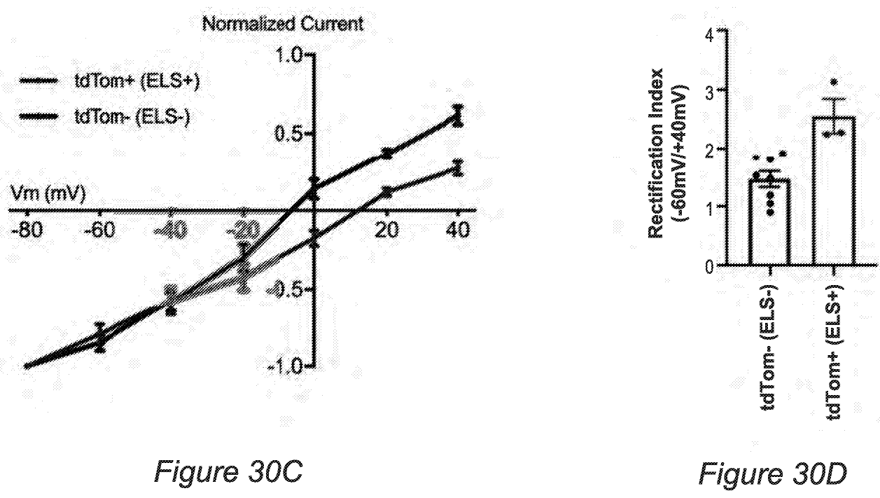

KA seizure ELS model: FosTRAP/FosGFP P10 mice will receive an i.p. injection of 4-OHT 1 hr prior to ELS induction. KA (2 mg/kg i.p.) or saline-vehicle will be administered and seizure responses recorded blind to group and scored for Racine stage, latency, and duration. Mice not reaching stage 5 tonic-clonic seizures will be eliminated from the study. Saline-vehicle treated littermates will be placed in the chamber for equivalent 90 min period. Robust DG, CA subfield staining shows a subset of tdTom positive (tdTom+) activated neurons (FIG. 30, which is not seen in the handling saline control, tdTom negative (tdTom−).

After ELS: Mouse brains will be harvested at either P15, P30 or P60 to follow changes in measurement parameters. Male and female mice will be used in equal numbers to identify gender differences. For each experiment for effects of ELS on later life, all three time points will be examined in each seizure model. For effects of ELS on LLS, a second seizure will be induced at P30 (KA model: 15 mg/kg or 20 mg/kg; PTZ model: 45 mg/kg) or P60 (KA model: 15 mg/kg or 25 mg/kg; PTZ model: 45 mg/kg) and in each case mice will be euthanized at 4 hrs post-LLS.

Power analyses: Both within-group variability and between-group differences will be measured for each outcome. Group size was estimated using power analyses (p=0.05; a=0.8, 30% difference), where n=12 will be used for experiments unless noted otherwise. We will require group sizes of at least 12 to determine differences from the normal distribution, and to avoid type I and type II errors. Statistical significance threshold will be p<0.05 and UPENN Biostats Core consulted for all analytic methods.

To Determine Whether Neurons Activated by ELS have Persistent, Life-Long, Alterations of Glutamate Receptor Function Associated with Impaired Synaptic Plasticity and Hyperexcitability Compared to Neurons from No-Seizure Control Mice.

Although ELS cause permanent decreases in plasticity and increased network excitability,59, 108 it has been challenging to find alterations at a single neuron level that underlie the long term deficits in synaptic plasticity seen after ELS. The inability to track seizure-induced changes over time has hampered our ability to identify new therapeutic targets. We believe that the FosTRAP/FosGFP model will reveal a subpopulation of cells that are activated by seizures, and that these cells will express permanent changes in AMPAR function and expression. We hypothesize that these neurons will have permanent evidence of hyperexcitability, greater AMPAR current inward rectification and evoked Ca²⁺ permeability than control neurons and surrounding unlabeled neurons. Given both NBQX and IEM-1460 showed disease-modifying efficacy after ELS, if these treatments affect any of the changes, this would support their nature and critical factors and warrant further investigation of AMPARs, and specifically GluA2-lacking AMPARs, as disease modifiers as well as justify further examination of the temporal evolution of other downstream changes. The primary focus is whether the cells that were unambiguously activated by ELS (TdTom+) exhibit altered physiological profiles throughout the lifespan, and to identify whether single neuron changes ultimately drive the impairments observed in later life at the network and in vivo level. We will record from age-matched no-seizure control mice as the “true” controls. While the surround tdTom− cells in seizure mice are not the main emphasis as it cannot be guaranteed they were not activated, we will still compare them to the tdTom+ cells to determine if they more closely resemble the ELS-tagged neurons or those from age-matched sham no-seizure control mice. All recorded cells will be biocytin-labeled for confirmation by IHC.

Do tdTom+ Neurons after ELS Show Later Alterations in Intrinsic and Synaptic Properties Compared to Those in the No Seizure Age-Matched Mice or to Surrounding tdTom− Neurons? do these Properties Change Across Time with Brain Maturation?

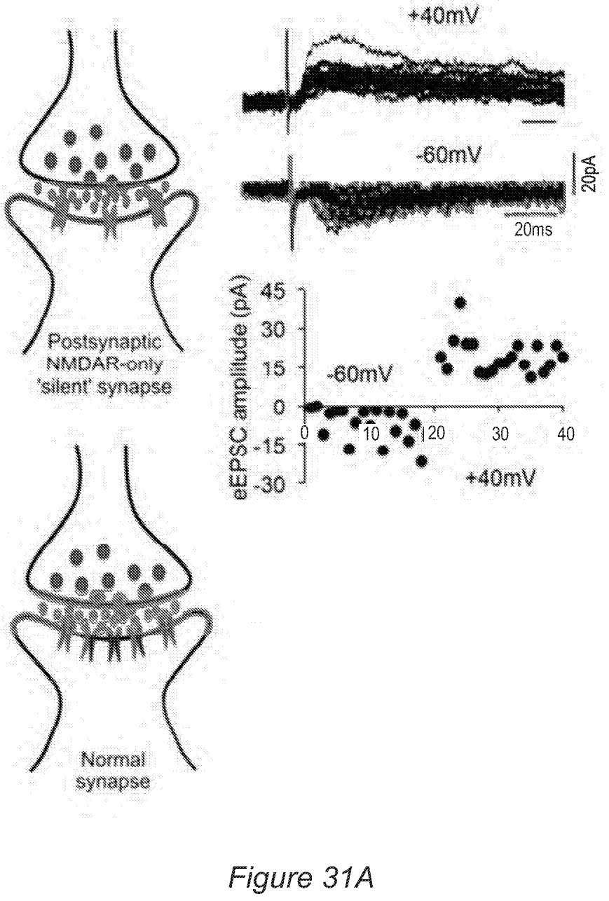

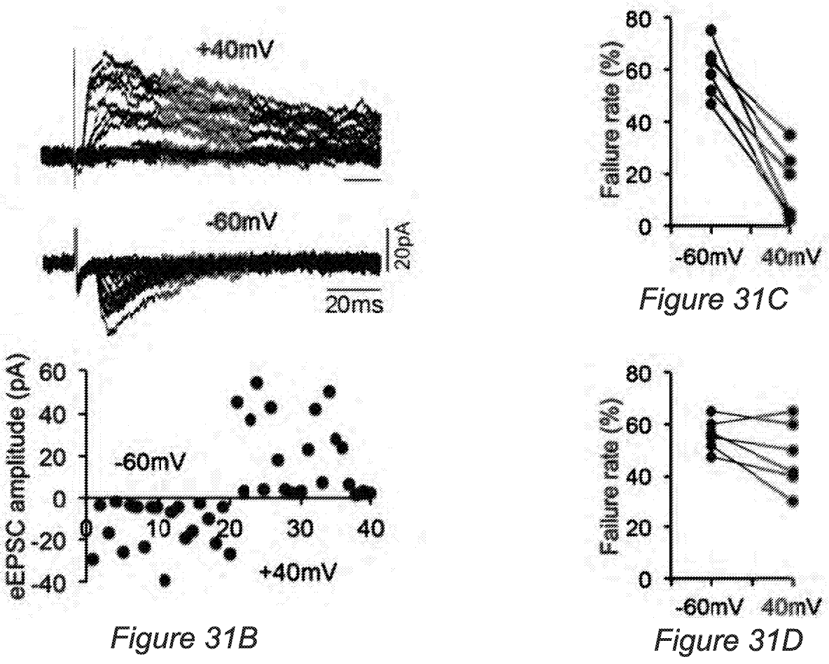

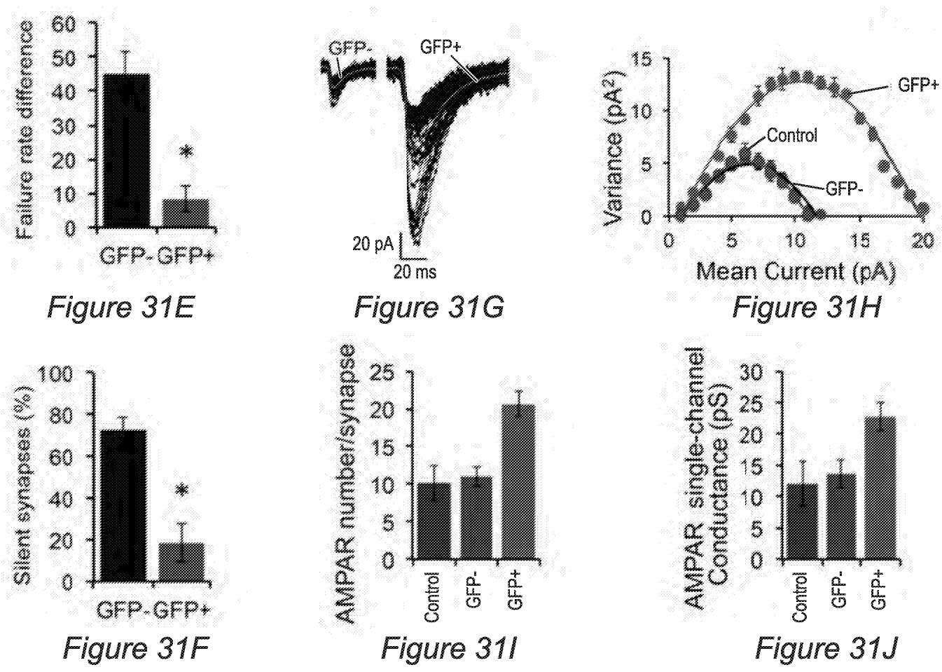

Methods: Brain slices will be prepared for electrophysiology from FosTRAP/FosGFP mice (n=12 mice/group, 2-3 slices/animal) at P15, P30, or P60 after ELS described above. Hippocampal slices will be prepared from KA-induced seizure and vehicle-control littermates for evaluation of CA1 pyramidal neurons.108 Auditory thalamocortical slices will be prepared from PTZ-injected and vehicle-control mice for evaluation of L4 pyramidal neurons as per our published protocols. We will record from neurons visualized by NIR-DIC and fluorescence microscopy to examine tdTom+ or tdTom− neurons (FIG. 31) in both treatment groups. Intrinsic properties will be evaluated in each group using whole-cell current-clamp recordings, specifically measuring resting membrane potential, input resistance, spike threshold, action potential parameters (i.e., amplitude, duration, rise and decay times), and input-output curves comparing spike numbers to current steps. Synaptic function will be evaluated using whole-cell voltage-clamp recordings to record and analyze differences in amplitude, frequency and distribution of spontaneous and miniature excitatory post-synaptic potentials (s/mEPSCs) and inhibitory post-synaptic potentials (s/mIPSCs). Evoked EPSC measures of paired-pulse ratio, NMDA:AMPA ratio, and AMPAR I-V plots will be performed as per our protocols. We will compare outcome measures from tdTom+ and tdTom− cells in each group, and across treatment and age groups.

Do tdTom+ Neurons after ELS Selectively Exhibit Diminished Plasticity or Silent Synapse Loss Compared to Those from the No-Seizure Age-Matched Mice or Surrounding tdTom− Neurons? do these Properties Change Across Time with Brain Maturation?

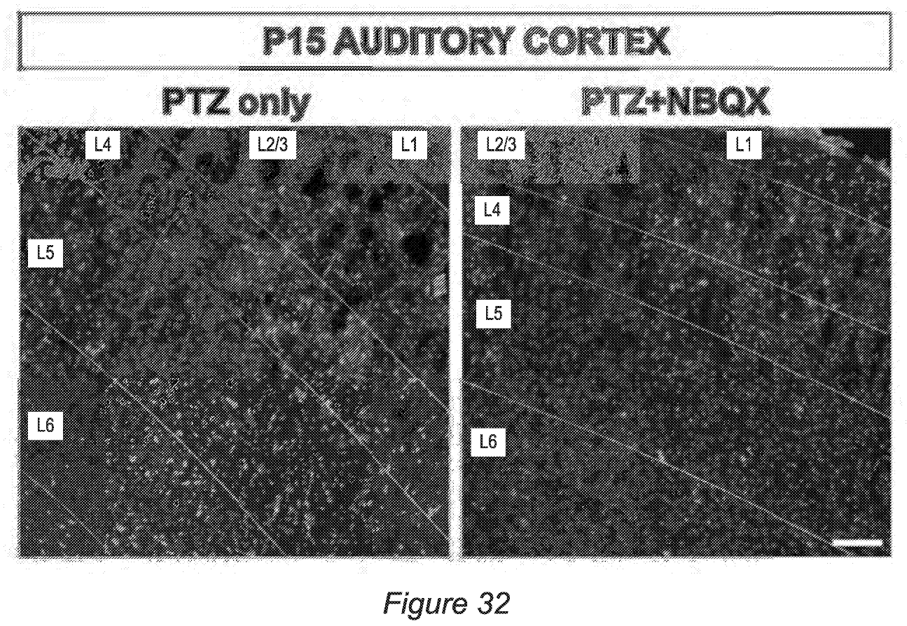

Every effort will be made to perform these experiments on the slices from those collected above. As per our recent work with hippocampal slices removed after ELS from FosGFP mice (FIG. 32) single cell evoked EPSC-LTP will be tested with pairing protocols at room temperature (to prevent washout at 30-32° C.), and cells will be held at 10 mV with 2 tetani (0.3 ms 100 Hz, separated). To evoke NMDA-dependent LTD, neurons will be clamped at −40 mV in cesium-based internal solution at room temperature with picrotoxin (60 uM), and a 5 Hz stimulation will be applied to the Schaffer collateral pathway for 3 min. Changes in access resistance will be monitored. LTP and LTD in tdTom+ and tdTom− neurons will be determined by comparing evoked EPSC amplitude pre- and post-pairing protocols. We will use our protocols of evoked EPSCs and failure rates to measure the ratio of silent to functional synapses in tdTom+ and tdTom− cells. Silent synapses are calculated by using a minimal stimulus intensity that results in a 50-60% evoked EPSC failure rate (˜0 pA post-synaptic response) while clamped at −60 mV. Cells will be then clamped at +40 mV and the failure rate calculated using the same intensity. The fraction of silent synapses will be calculated: (1−ln(F₋₆₀)/ln(F₊₄₀)).

Does AMPAR Antagonist Treatment Early Post-ELS Attenuate the Altered Electrophysiological Responses in the Different Neuron Populations? does this Change Over Time?

Seizures will be induced as described above. For the KA model, beginning 1 hour after KA seizure, mice will receive doses of either NBQX (20 mg/kg i.p.), 60 IEM 1460 (10 mg/kg i.p.), or saline every 12 hrs×4 doses as per prior protocols with ELS. For the PTZ model, we will follow our prior protocol, and deliver the NBQX (20 mg/kg i.p.), IEM 1460 (10 mg/kg i.p.) or saline at 1 hr post seizure on P9, P10, and P1189 (FIG. 33). Mice will be euthanized at P15, P30, or P60. Two sets of groups will be prepared (n=12/group): the first for perfusion and subsequent IHC to determine whether treatment decreases the number of tdTom+ cells. Confocal imaging of at least 2 fields/section and 3 sections/mouse in defined areas of hippocampal CA subfields and dentate gyrus (hippocampus: Bregma −1.46 to −2.70 mm (interaural 2.34 mm-1.1 mm) or primary auditory cortex (Bregma −2.18 mm to −3.64 mm (interaural 1.5 mm to 0.16 mm) will be used. MAP2, Iba-1, GFAP, and GAD67 will be used to confirm cell identity. Cell counts will be performed using ImageJ software and group differences analyzed by 2-way ANOVA/Bonferroni correction. Multivariate linear and logistic regression will be used to evaluate effects of age, gender, treatment groups and seizure severity in ELS. Our preliminary results show a decrease in the number of tdTom+ cells in NBQX-treated PTZ ELS mice (FIG. 33). The second group will be prepared for electrophysiology experiments in both tdTom+ and tdTom− neurons, focusing on silent synapses/AMPAR currents.

Data Analysis: For each animal, we will obtain recordings from at least one tdTom+ and tdTom− pyramidal neuron (within 50 μm) in the same cell body layer and slice, and a similar number in slices from the no-seizure controls. All data will be expressed as mean±SE. For two-group comparisons, statistical significance will be assessed using two-tailed Student's unpaired or paired t test for normally distributed data or non-parametric two-tailed Mann-Whitney U test for data not distributed normally. Multi-group comparisons will be performed using 1 and 2-way ANOVA for normally distributed data or Kruskal-Wallis nonparametric tests. For comparison across groups with unequal variance, the unequal-variance t-test using an unpooled SE will be performed. For each dataset, a 1-way ANOVA will examine gender effects. The Shapiro-Wilk test will be used to test for normality, and Levene's method will be used to test for equal variance.

Results: We expect tdTom+ neurons to have different physiological properties compared to same slice surround neurons and neurons in identical populations from no-seizure control mice and at all ages tested. The differences may change with age and may reveal new stage-specific therapeutic targets. We do not expect ELS-induced changes in intrinsic properties or paired-pulse responses based on prior results, the latter consistent with our hypothesis that these changes are primarily postsynaptic. At P15, as per pilot data in FosGFP mice (FIG. 28, 32) we expect to see robust changes in synaptic function in tdTom+ neurons in both CA1 and L4, as in our prior work (increased s/mEPSC amplitudes, inwardly rectifying AMPA currents, decreased silent synapse fraction and impaired plasticity) compared to vehicle, no-seizure control neurons and surrounding tdTom− neurons. We also expect that P15 no-seizure and tdTom− neurons will show immature AMPAR characteristics in patterns that increase E:I ratio79 compared with later P30 and P60 ages as this is a pattern of normal development, but to be less exaggerated compared with tdTom+ cells. However, at P30 and 60 after this developmental window closes, we would expect to see persistence of these changes in the tdTom+ neurons. We are aware that the tdTom+ cells may not represent the entire population of ELS activated cells, and therefore we expect to also see a gradient of abnormalities in tdTom− CA1 or L4 neurons, as these include populations that were either not activated, or were engaged in the seizure but remained unlabeled due to unsuccessful Cre-mediated recombination. If the persistent upregulation of AMPAR function in tdTom+ at P30 and P60 has functional consequences, we would expect to see those neurons exhibiting diminished synaptic plasticity. Another possibility is that the impaired plasticity might be due to an excessive homeostatic downregulation of AMPAR synaptic function, and this would be indicated if EPSC amplitude and frequency were decreased, similar to the transient phenomenon we observed within 48 hrs of ELS due to activity dependent increases in PLK2.88 If over time, unlabeled neurons have no abnormalities, this would indicate that the unlabeled neurons may have not been activated, and experienced the normal downregulation of excitability and increases in GluA2, with linear rectification, while the tdTom+ cells may have had this development dysregulated and continue to have impaired plasticity. If tdTom− cells are also abnormal this would indicate they have been indeed activated by the initial seizure, or secondarily recruited over time, and this would open up a window for therapy. We may see GFP expression in tdTom+ or tdTom− cells due to spontaneous excitability at P30 or P60, suggesting a secondary spread of network hyperexcitability. Given our prior results, we expect that early post-ELS treatment with NBQX may decrease the number of tdTom+ cells, as 4-OHT enables “TRAPing” for up to 6 hrs after injection and hence if there is de novo c-fos activation occurring 3-4 hrs after seizure induction, NBQX might block this very induction. However, we hypothesize that most c-fos induction is within the first hour, before we give the NBQX, so it is also possible that we will only see subtle decreases in the numbers of tdTom+ neurons compared to ELS with vehicle. Nevertheless, as we hypothesize that critical changes in neuronal function are downstream of initial AMPAR activation, we would expect that NBQX would significantly attenuate most changes in the tdTom+ cells and surround neurons at P15, P30 and P60. As we also hypothesize that GluA2-lacking receptors are particularly responsible for triggering these downstream changes, we expect that IEM-1460 treatment will be equally or even more effective as NBQX in attenuating the ELS-induced alterations. Some variables may trend for enhanced responses in male mice, and we will extend experiments to confirm these changes.

Determining Whether Neurons Activated by ELS have Persistent Alterations of Gene and Protein Expression Related to Glutamate Receptor Function Compared to Neurons from No-Seizure Control Mice.

We hypothesize that there are critical changes in the transcriptome occurring over time specific to ELS-activated tdTom+ neurons versus no-seizure control and surround tdTom− neurons. FosTRAP/FosGFP mice will be euthanized at P15, P30 and P60 after ELS. Nuclear mRNA expression in tdTom+ cells will be compared to no-seizure control and surround cells using FACS of neurons in brain regions of interest to examine nuclear RNA. We will examine cellular and dendritic RNA expression using novel LT-TISA technique. IHC will examine specific changes in synaptic protein expression.

Are there Changes in Nuclear RNA Selectively in Activated Neuronal Populations, and how do they Change Over Time?

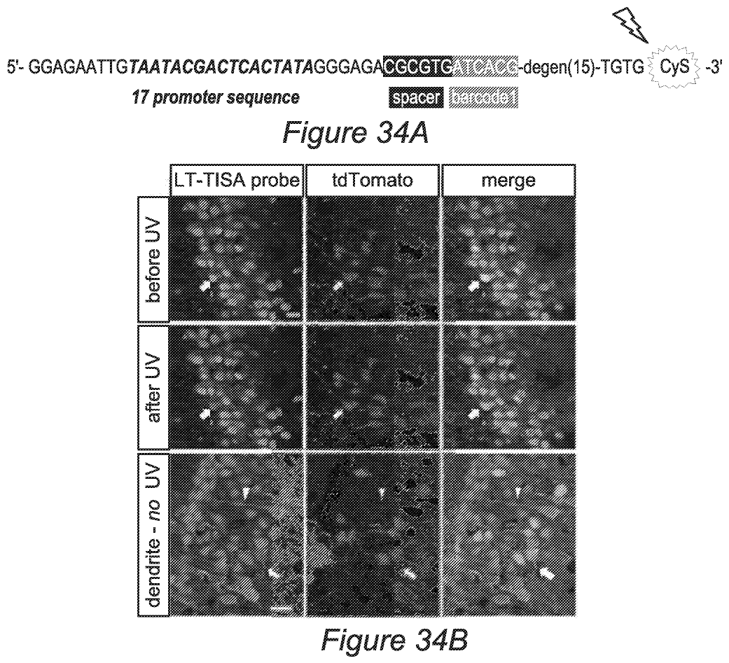

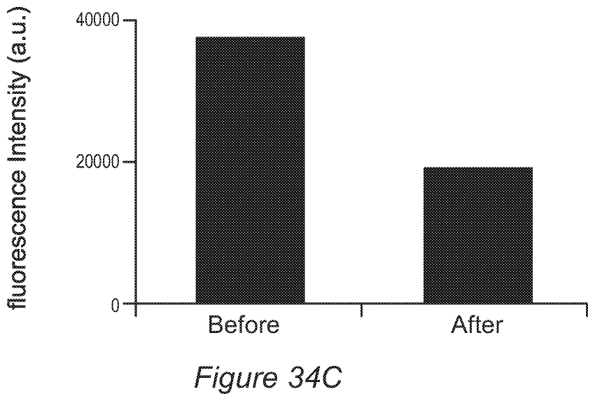

Methods: ELS will be induced in FosTRAP/FosGFP mice as above, and mice will be euthanized at P15, P30 and P60 (n=12/group for KA or PTZ versus vehicle). For the KA model, we will harvest hippocampus; for the PTZ model we will harvest cortex. Nuclei will be isolated from fresh cortical and hippocampal tissue for FACS, with 2-3 mice pooled per sample (with 3 biological replicates). Using published protocols and those from Eberwine, tissue will be dounce homogenized, layered onto a sucrose cushion and ultracentrifuged. Resuspended nuclei will be incubated with RNase inhibitor and stained with NeuN conjugated 647 and DAPI (for singlet detection). Cells will be sorted for tdTom+ neuronal nuclei vs. tdTom− neuronal nuclei at the UPENN Flow Cytometry and Cell Sorting Facility using a BD Sciences Aria cell sorter, similar to our FACS with the FosGFP mouse tissue (FIG. 34). Total nuclear RNA will be isolated from sorted cells with RNeasy Micro isolation kit (Qiagen) and stored at −80° C. RNA-seq library will be prepared with TruSeq Total RNA Library Prep Kit and Ribo-Zero (Illumina). Equal amounts of multiplexed libraries will be mixed and subjected to paired-end sequencing on the Illumina HiSeq 2000/25000 platform at Penn's Next Generation Sequencing Core. RNA-seq data will be mapped to the mouse Ensembl genome by STAR. Differentially expressed genes will be determined based on the final read count from the total number of read pairs mapped onto a gene, with functional annotation using DAVID gene ontology and gene set enrichment analysis performed using Mouse GO Gene Set Release.

Does Dendritic RNA Related to Glutamate Receptor-Mediated Signaling Change Over Time in Neurons Activated by an ELS Compared to No-Seizure Control Neurons and Those in the Surround?

Methods: Using mice prepared as above, we will analyze single dendritic NA transcriptome selectively from differentially activated neuronal populations. Lightening Terminator-Transcriptome in situ analysis (LT-TISA) will be performed in collaboration with Dr. Jim Eberwine. LT-TISA utilizes a novel multifunctional oligonucleotide (FIG. 35) that anneals randomly to single-stranded RNA via a 15-nucleotide degenerate sequence. A dideoxynucleotide with a Cy5 fluorescent moiety on the 3′ end renders the LT-TISA oligonucleotide inactive for in situ cDNA synthesis unless photoactivated. Targeted photoactivation with a UV laser will cause the removal of Cy5, whereby the free 3′-OH that is formed acts as an in situ primer for copying of the annealed mRNA into cDNA. The LT-TISA oligonucleotide also contains a T7 RNA polymerase promoter site and barcode to facilitate amplification and subsequent analysis.

As per Eberwine protocols, FosTRAP/FosGFP mice will be perfused in RNase free conditions at the post-ELS timepoints and brains sectioned at 60 μm. MAP2 immunostaining will be performed on free-floating sections to label neuronal dendritic processes. Following staining, LT-TISA probes will be loaded onto the brain slices whereby imaging and photoactivation will be performed using a Zeiss 710 Meta confocal microscope. Loading is confirmed (Cy5 fluorescence). TdTom+ dendrites and tdTom−/MAP2+ dendrites loaded with the LT-TISA probe will be selectively irradiated in CA1 or L2/3 auditory cortex (dendritic fields of L4) using a 405-nm UV laser for removal of the lightning terminator site (60% power, 6.30 μs per pixel; activation is confirmed with loss of Cy5 fluorescence) (FIG. 35). Slices will be incubated in reverse transcriptase and dNTP for first-strand cDNA synthesis, and cDNA will be harvested from single dendrites using micro glass pipettes. The isolated cDNA will be copied into double-stranded DNA and amplified using T7 RNA polymerase, followed by library construction, sequencing, and genome alignment, as per Expt 2a. Approximately 10-20 dendrites (mix of tdTom+vs. tdTom−/MAP2+) will be isolated in auditory cortex and hippocampus of a given slice with 3 biological replicates.

Are GluA2 Protein Levels Differentially Expressed Over Time in Neurons Activated by an ELS Compared to No-Seizure Control Neurons and Those in the Surround?

Methods: We will use sections from above (saline, 4-OHT-treated, ELS and 4-OHT-treated no seizure mice), using our protocol to stain extracellular GluA2 or GluA1, presynaptic synapsin, and MAP2 dendrites, where the distribution and co-localization of GluA2 or GluA1 with synapsin will be examined in tdTom+ or tdTom− dendrites in both groups (FIG. 36). Confocal images will be obtained at 63× in hippocampal CA1 or L2/3 auditory cortex (dendritic field of L4) localized based on the Paxinos75 and Allen Developing mouse brain atlases. The fraction of GluA2/synapsin and GluA1/synapsin co-localized puncta in tdTom+ and tdTom−/MAP2+ dendrites will be compared, as well as total dendritic expression (regardless of tdTom expression after ELS compared to age-matched controls). ImageJ will be used to quantify confocal images at 63×.

Does AMPAR Antagonist Treatment Early Post-ELS Attenuate Long-Term Alterations in RNA and Protein Levels Related to Glutamate-Mediated Neuronal Excitability and GluA2 Dysfunction?

Methods: Mice will be treated as above with IEM1460 (10 mg/kg), NBQX (20 mg/kg) or saline. P30 and P60 brains will be harvested for nuclear FACS/RNA-seq, dendritic LT-TISA, and sections from above (vehicle & AMPAR antagonist-treated ELS and control mice) for GluA2/GluA1-synapsin co-localization as above.

Data analysis and outcome measures. Changes in gene expression between control, tdTom+ and tdTom− cells will be analyzed similarly to established methods. Data will be analyzed with established methods by Eberwine's lab and a biostatistician will be consulted. For both LT-TISA and IHC co-labeling, we will obtain images of tdTom+ and tdTom− processes in the same cell body layer and slice, to compare with region-matched no-seizure control slices. Data will be expressed as mean±SE and compared using 1 and 2-way ANOVA for multiple groups and gender with Bonferroni or Mann-Whitney to analyze differences between control, tdTom+ and tdTom− cells and processes. Correlations will be examined using logistic and linear regressions.

Results: Given the long lasting behavioral and plasticity changes elicited by ELS, we expect that ELS mice differentially express genes compared to vehicle-saline controls, regardless of tdTom expression as we and others have shown activity-related transcriptome changes following acutely after seizures. Within mice that have had ELS, given our pilot data in FosGFP mice (FIG. 34) we expect to see more pronounced transcriptome changes in tdTom+ neuronal nuclei compared to tdTom− nuclei using RNA-seq from FACS. Since ELS evoke excitatory activity in the brain, we anticipate that gene ontology analysis will indicate differential expression in gene families related to activity-dependent Ca2+ pathways, plasma membrane, synapse, ion channels/transporters, protein phosphorylation, mitochondria, and more. Dendritic mRNA transcriptome analysis (LT-TISA) in tdTom+ dendrites will likely exhibit more pronounced or differentially regulated sets of changes compared to nuclear RNA, related to synaptic regulation and localization, given known activity-dependent trafficking and translation of mRNAs within neuronal processes for local regulation. While we will examine overall gene ontology of differentially expressed genes, we will specifically examine transcripts that are related to AMPAR regulation (e.g., PSD95, STEP, LRRTM, TRIO, neurogranin, FXR1P, FXR2P34). Furthermore, given our observed decreases in synaptic GluA2 48 h post P10 hypoxic seizure, we expect reduced GluA2-synapsin and increased GluA1-synapsin colocalization in tdTom+ dendrites compared to tdTom− dendrites and those from naïve controls; however, it is possible that a gradient exists where tdTom− dendrites have altered synaptic expression, just not as robustly as tdTom+. AMPAR antagonists are likely to reverse many of the activity-related transcriptome changes from ELS and will serve as a tool to validate causality of genes associated with AMPARs change. Some variables may trend for enhanced responses in male mice, and we will extend experiments to confirm these changes.

Although we observe robust tdTom expression following ELS, if we cannot isolate enough tdTom+ nuclei from the hippocampus for FACS and subsequent RNA-seq, we will pool more brains together, or do single-cell LT-TISA selectively from hippocampal CA1 cell bodies/cytosol. Similarly, if not enough RNA can be obtained from single dendritic isolations using LT-TISA, we can pool together first strand cDNA across multiple tdTom+ and tdTom− dendrites rather than evaluating at a single cell level. Other alternatives include laser capture techniques applied to activated dendrites.

To Determine Whether Neurons Activated by ELS are Differentially Affected by a Second Later Life Seizure (LLS) in Adulthood Compared to Neurons from No-Seizure Control Mice.

In addition to causing impaired network plasticity, ELS enhance neuronal excitability and the susceptibility for later life seizures. Thus, it is crucial to identify whether neurons originally activated during the ELS have differential responses to a second LLS relative to a no-ELS control mouse and surround neurons in the same slice only activated by the LLS. Using the ELS paradigm described above, FosTRAP/FosGFP mice, including no-seizure and saline controls, will be subjected to an additional LLS at P30 or P60, and where brains will be harvest at 4 hrs post seizure (FIG. 29). This paradigm optimizes the Fos-GFP expression whereby neurons activated by the LLS will be identified by presence of nuclear GFP. Thus, in conjunction with tdTom labeling from the ELS, we will be able to track subpopulations of neurons: those activated by ELS only will be tdTom+ only (red) and those activated by both the ELS and LLS will be tdTom+/GFP+(yellow) (FIGS. 29, 37). Hence, we can analyze the differential recruitment properties of tdTom+ neurons to seizures at a later stage. We will also examine how early life AMPAR antagonist treatment blocks subsequent responses to LLS. Collectively, tracking how ELS modifies neuronal populations long term and in response to multiple seizure events will allow us to identify therapeutic targets for epilepsy cognitive co-morbidities (and epileptogenesis). These experiments will only include IHC and electrophysiology evaluation, as the pursuit of gene expression by FACS and TISA will be subject of a future study dependent upon the success of these experiments. Taken together with the earlier experiments, we can examine whether impaired plasticity is related to neuronal hyperexcitability, or whether these are separable.

Does a Seizure in Later-Life Reactivate the Same Population of Neurons in FosTRAP/FosGFP Mice to Those Originally Activated by a Prior ELS?

Methods: LS and LLS will be induced and mice perfused at P30 or P60 (n=12/group), per our methods.60 FosTRAP/FosGFP mice will receive the same ELS paradigm as in Aims 1 and 2, including a no-seizure group. All groups (n=12-14/group including both sexes) will undergo a second LLS at P30 (20 mg/kg KA or 45 mg/kg PTZ) or P60 (25 mg/kg KA or 45 mg/kg PTZ), where LLS seizure scores and latency to each Racine stage will be recorded. Brains will be harvested 4 hrs post LLS, as FosGFP expression lasts 4-6 hrs after tonic-clonic seizures,7 while tdTom+ expression lasts >1 year. Confocal microscopy (20 μm sections) will assess neurons activated by ELS and LLS in hippocampus and auditory cortex from the KA and PTZ model, respectively. Using the same stereotactic locations as above, tdTom+ only and co-labeled tdTom+/GFP+ neurons will be counted to assess the distribution of cells activated in each of the seizure events. Neuronal identities of activated cells with be confirmed with IHC of NeuN or GAD67. As delayed neuronal death can occur after seizures in P30 and P60, an additional group (n=12) of ELS/LLS mice will be euthanized 48 hrs after LLS. All sections will be stained with TUNEL and FluoroJade B staining to isolate neurons in the process of dying. For all comparison of IHC markers counts between groups, we will use 2-way ANOVA (Bonferroni correction). Multivariate linear and logistic regression will evaluate effects of age, gender and seizure severity.

Do Neurons Activated by Both ELS and LLS Exhibit Different Synaptic or Intrinsic Properties Compared to Those Activated by Only the ELS or LLS Events?

Methods: P30 and P60 FosTRAP/FosGFP mice will be subjected to ELS and LLS paradigms (n=12/group), and slices prepared at 4 hrs post LLS or saline no-seizure for whole-cell recordings of tdTom+/GFP+ and tdTom+/GFP− neurons (2-3 slices per mouse). We will compare intrinsic properties, s/mEPSC/IPSC amplitude, frequency and number, and evoked AMPAR I-V rectification between groups/cell types. We will compare these data for age-matched comparisons, and correlate with LLS seizure severity.

How does LLS Impact Synaptic Plasticity of Neurons after ELS?

Methods: Using the same brain slice preparations as above, and the same protocols, outcome measures and analyses as in Expt. 2b we will examine for silent synapses, LTP and LTD in each cell type (tdTom+, GFP+, tdTom+/GFP+, tdTom+/GFP−). We will identify how the capacity for plasticity is altered, and whether a gradient exists in the severity of impairment following the heterogeneous activation from LLS and/or ELS, and correlate with LLS seizure severity.

Does ELS AMPAR Antagonist Post-Seizure Treatment Attenuate the Altered Electrophysiological Responses in the Different Neuron Populations?

Methods: As described earlier, treatment with AMPAR antagonists immediately following ELS ameliorates later life consequences. Using the NBQX and IEM-1460 ELS paradigm, along with no-seizure and saline-controls, we will compare whether treatments attenuate the effect of LLS after ELS. First, we will use IHC (as in Expt 1c) to analyze if the number of tdTom+ cells are decreased following LLS in mice with post ELS AMPAR antagonist treatment, as suggested by our preliminary data (FIG. 37). We will assess whether AMPAR antagonist treatment reduces the number of cells reactivated by the LLS by quantifying tdTom+/GFP+ cells. Another cohort of post-ELS treated mice will be compared to above to assess whether circuit dysfunction and aberrant neuronal excitability is attenuated with AMPAR antagonists post seizure in the different neuron “types”.

Results: As the c-fos driven GFP expression occurs following a second seizure, we expect that a greater % of originally activated neurons (tdTom+) will show GFP activation (yellow) However, the total number of GFP+(green) only cells will be greater, due to the overall increase in synaptic connections and recruitment of greater neuronal population in the mature brain than P9-11. We expect inward rectification, silent synapses, LTP and LTD impairments will be greatest in yellow tdTom+/GFP+ neurons, and greater at P60 than P30. In the mice killed at 48 hrs post LLS, if we see greater death in tdTom+ cells compared to GFP+ only cells, it would suggest altered glutamate function enhances status-induced cell death, but if they are less affected, this would suggest homeostatic downregulation of GluRs as a mechanism of impaired plasticity. These outcomes would help us understand whether cognitive impairment is separable from neuronal excitability. We also expect that IEM-1460 treatment post ELS will be more effective than NBQX in blocking altered responses to LLS.

What is claimed is:

1. A method for preventing or reducing the risk of developing a neurological disorder consequent to early-life seizure or hypoxic encephalopathy, comprising administering to a subject having had early-life seizure or hypoxic encephalopathy, an effective amount of an antagonist of CP-AMPAR, wherein CP-AMPAR lacks a GluA2 subunit.

2. The method of claim 1, wherein the antagonist is IEM1460

3. The method of claim 1, wherein the antagonist is systemically administrable.

4. A method for treating a subject suffering from enhanced CP-AMPAR function or expression, said method comprising administering an effective amount of an antagonist of CP-AMPAR, wherein CP-AMPAR lacks a GluA2 subunit to the subject.

5. The method of claim 4, wherein the subject is at a developmental stage having a predominance of GluA2-lacking AMPARs.

6. The method of claim 4, wherein the subject has an early-life seizure.

7. The method of claim 4, wherein the subject has hypoxic encephalopathy.

8. The method of claim 4, wherein the subject has a CDKL5 disorder,

9. The method of claim 4, wherein the subject further has one or more neurologic disorder.

10. The method of claim 9, wherein the one or more neurologic disorder is infantile spasms, Lennox Gastaut syndrome, Rett Syndrome, West Syndrome, and autism.

11. The method of claim 4, wherein the subject has epilepsy.

12. The method of claim 4, wherein the subject has an autism spectrum disorder.

13. The method of claim 4, wherein the subject has dementia.

14. The method of claim 4, wherein the subject has a neurodevelopmental delay disorder.

15. The method of claim 4, wherein the subject has a traumatic brain injury.

16. The method of claim 4, wherein the subject has a stroke.

17. The method of claim 4, wherein the seizure is post-natal.

18. The method of claim 4, wherein the seizure is from 3 to 6 months after birth.

19. The method of claim 4, wherein the antagonist is administered from between immediately post-seizure to 6 months post-seizure.

20. The method of claim 19, wherein the antagonist is administered immediately post-seizure.

21. The method of claim 19, further comprising administering an L-type voltage gated Ca²⁺ channels (LT-VGCC) blocker.

22. The method of claim 21, wherein the LT-VGCC blocker is nimodipine.

23. The method of claim 4, wherein administration of the antagonist either delays later-life epilepsy.

24. The method of claim 4, wherein administration of the antagonist further either delays or reduces incidence of later-life epilepsy.

25. The method of claim 4, wherein administration of the antagonist further delays or reduces incidence of autism spectrum disorders.

26. A method for treating a subject suffering from a disease associated with phosphorylation of the transcriptional regulator methyl CpG binding protein 2 (MeCP2), comprising: administering an effective amount of an antagonist of a calcium permeable, AMPA subtype glutamate neurotransmitter receptor (CP-AMPAR), wherein CP-AMPAR lacks a GluA2 subunit; or an antagonist of an L-type voltage gated Ca²⁺ channels (LT-VGCC) blocker; or both.

27. The method of claim 26 wherein the CP-AMPAR antagonist is systemically administrable.

28. The method of claim 26 wherein the LT-VGCC antagonist is systemically administrable.

29. The method of claim 26, wherein the antagonist of the CP-AMPAR is IEM1460.

30. The method of claim 26, wherein the LT-VGCC blocker is nimodipine.

31. The method of claim 26, wherein the subject is at a developmental stage having a predominance of GluA2-lacking AMPARs.

32. The method of claim 26, wherein the subject has an early-life seizure.

33. The method of claim 26, wherein the subject has hypoxic encephalopathy.

34. The method of claim 26, wherein the subject has a CDKL5 disorder,

35. The method of claim 26, wherein the subject further has one or more neurologic disorder.

36. The method of claim 35, wherein the one or more neurologic disorder is infantile spasms, Lennox Gastaut syndrome, Rett Syndrome, West Syndrome, and autism.

37. The method of claim 26, wherein the subject has epilepsy.

38. The method of claim 26, wherein the subject has an autism spectrum disorder.

39. The method of claim 26, wherein the subject has dementia.

40. The method of claim 26, wherein the subject has a neurodevelopmental delay disorder.

41. The method of claim 26, wherein the subject has a traumatic brain injury.

42. The method of claim 26, wherein the subject has a stroke.

43. The method of claim 26, wherein the seizure is post-natal.

44. The method of claim 26, wherein the seizure is from 3 to 6 months after birth.

45. The method of claim 26, wherein the antagonist is administered from between immediately post-seizure to 6 months post-seizure.

46. The method of claim 45, wherein the antagonist is administered immediately post-seizure.

47. The method of claim 45, wherein the blocker is administered immediately post-seizure.

48. The method of claim 45, wherein the blocker and the antagonist are administered immediately post-seizure.

49. The method of claim 26, wherein administration of the antagonist delays later-life epilepsy.

50. The method of claim 26, wherein administration of the antagonist further either delays or reduces incidence of later-life epilepsy.

51. The method of claim 26, wherein administration of the antagonist further delays or reduces incidence of autism spectrum disorders.

52. The method of claim 26, wherein administration of the blocker delays later-life epilepsy.

53. The method of claim 26, wherein administration of the blocker further either delays or reduces incidence of later-life epilepsy.

54. The method of claim 26, wherein administration of the blocker further delays or reduces incidence of autism spectrum disorders.

55. The method of claim 26, wherein administration of the antagonist and blocker delays later-life epilepsy.

56. The method of claim 26, wherein administration of the antagonist and blocker further either delays or reduces incidence of later-life epilepsy.

57. The method of claim 26, wherein administration of the antagonist and blocker further delays or reduces incidence of autism spectrum disorders.
